(19) United States
(12) Patent Application Publication
(10) Pub. No.: US 2026/0182946 A1
(43) Pub. Date: Jul. 2, 2026

(54) SYSTEMS, METHODS, AND STORAGE MEDIA FOR BREAST IMAGING

(71) Applicant: SHANGHAI UNITED IMAGING HEALTHCARE CO., LTD., Shanghai (CN)

(72) Inventors: Xiaoke WANG, Shanghai (CN); Jia YANG, Shanghai (CN); Hailiang HUANG, Shanghai (CN)

(73) Assignee: SHANGHAI UNITED IMAGING HEALTHCARE CO., LTD., Shanghai (CN)

(21) Appl. No.: 19/433,183

(22) Filed: Dec. 26, 2025

(30) Foreign Application Priority Data
Dec. 26, 2024 (CN) 202411944636.1

(51) Int. Cl. A61B 6/50; A61B 6/00; A61B 6/04; A61B 6/46

(52) U.S. Cl. CPC A61B 6/502; A61B 6/0414; A61B 6/461; A61B 6/488; A61B 6/5217

(57) ABSTRACT

A method for breast imaging is provided. The method comprises controlling a breast scanning device to compress a target breast to a compressed state and utilizing a pressure sensor provided on the breast scanning device to acquire a pressure distribution map of the target breast in the compressed state; obtaining a reference identification result by identifying one or more target tissues of the target breast in the pressure distribution map, the one or more target tissues comprising at least one of a lesion tissue or a glandular tissue; determining, based on the reference identification result, one or more target scanning parameters; and controlling, based on the one or more target scanning parameters, the breast scanning device to perform a target scan on the target breast to acquire a target scanning image of the target breast.

### CROSS-REFERENCE TO RELATED APPLICATIONS

This application claims priority to Chinese Patent Application No. 202411944636.1 filed on Dec. 26, 2024, the contents of each of which is hereby incorporated by reference.

### TECHNICAL FIELD

The present disclosure relates to the field of medical imaging technology, and in particular, to a method, a system, and a storage medium for breast imaging.

### BACKGROUND

Breast cancer is a common cancer affecting women's health. Early detection, diagnosis, and treatment of breast cancer can significantly reduce mortality. Medical imaging examination is an important tool for breast cancer screening and diagnosis, among which mammography is a commonly used method for diagnosing breast diseases. The process of breast imaging involves multiple steps, such as breast positioning, scanning parameter setting, scanning, and image interpretation by physicians. In existing methods for breast imaging, these steps rely heavily on manual operations. This results in low scanning efficiency and a high workload for physicians, and makes the scans prone to errors due to inaccurate parameter settings, ultimately affecting scan quality.

Therefore, a method, a system, and a storage medium for breast imaging are provided, which can improve the efficiency and accuracy of breast imaging.

### SUMMARY

A method for breast imaging is provided. The method is implemented on a computing device comprising at least one processor and at least one storage device. The method comprises: controlling a breast scanning device to compress a target breast to a compressed state and utilizing a pressure sensor provided on the breast scanning device to acquire a pressure distribution map of the target breast in the compressed state; obtaining a reference identification result by identifying one or more target tissues of the target breast in the pressure distribution map, the one or more target tissues comprising at least one of a lesion tissue or a glandular tissue; determining, based on the reference identification result, one or more target scanning parameters; and controlling, based on the one or more target scanning parameters, the breast scanning device to perform a target scan on the target breast to acquire a target scanning image of the target breast.

In some embodiments, the one or more target scanning parameters comprise a target exposure parameter, the method further comprises obtaining a pre-scanning image of the target breast, the pre-scanning image being acquired by performing a pre-scan on the target breast in the compressed state using the breast scanning device. The target exposure parameter is determined by: determining, based on the reference identification result, one or more target regions corresponding to the one or more target tissues in the pre-scanning image; and determining, based on the one or more target regions and a pre-exposure parameter corresponding to the pre-scanning image, the target exposure parameter.

In some embodiments, the determining, based on the one or more target regions and a pre-exposure parameter corresponding to the pre-scanning image, the target exposure parameter comprises: determining an average grayscale value of the one or more target regions; determining, based on the average grayscale value and a target grayscale value of the target scanning image, a relationship multiplier; determining, based on the pre-exposure parameter corresponding to the pre-scanning image and the relationship multiplier, the target exposure parameter.

In some embodiments, the one or more target tissues comprise the glandular tissue, the lesion tissue, and an adipose tissue, and the reference identification result comprises a lesion identification result, a glandular identification result, and an adipose identification result. The determining, based on the reference identification result, one or more target scanning parameters comprises: determining, based on the lesion identification result, the glandular identification result, and the adipose identification result, a breast density of the target breast; and determining, based on the lesion identification result and the breast density, the one or more target scanning parameters, the one or more target scanning parameters comprising at least one of a target compression parameter, a target scanning angle, or a target exposure parameter.

In some embodiments, the controlling, based on the one or more target scanning parameters, the breast scanning device to perform a target scan on the target breast comprises: adjusting, based on the target compression parameter, a compression to the target breast applied by the breast scanning device; controlling, based on the target scanning angle and the target exposure parameter, the breast scanning device to perform the target scan on the target breast.

In some embodiments, the identification of the lesion tissue is performed based on a glandular threshold, the glandular threshold being determined by: determining a plurality of subregions in the pressure distribution map and pressure values of the plurality of subregions; determining, based on the pressure values of the plurality of subregions, a plurality of candidate subregions from the plurality of subregions; and determining, based on pressure values of the plurality of candidate subregions, the glandular threshold.

In some embodiments, the identification of the lesion tissue is performed based on a glandular threshold, the glandular threshold being determined by: obtaining a dynamic pressure distribution map acquired by the pressure sensor during a process of compressing the target breast to the compressed state; determining, based on the dynamic pressure distribution map, lesion depth information; and determining, based on an installed position of the pressure sensor and the lesion depth information, the glandular threshold.

In some embodiments, the identification of the lesion tissue is performed based on a glandular threshold, the glandular threshold being determined by: obtaining a dynamic pressure distribution map acquired by the pressure sensor during a process of compressing the target breast to the compressed state; determining, based on the pressure distribution map, a first pressure feature; determining, based on the dynamic pressure distribution map, a second pressure feature; processing the first pressure feature and the second pressure feature using a threshold determination model to determine the glandular threshold, the threshold determination model being a trained machine learning model.

In some embodiments, before acquiring the pressure distribution map, the method further comprises: utilizing the pressure sensor to acquire an initial pressure distribution map of the target breast; performing, based on the initial pressure distribution map, a verification on a current positioning of the target breast; and in response to determining that the current positioning passes the verification, designating the initial pressure distribution map as the pressure distribution map.

In some embodiments, the method further comprises: obtaining a dynamic pressure distribution map, the dynamic pressure distribution map being acquired by the pressure sensor during a process of compressing the target breast to the compressed state; determining, based on the dynamic pressure distribution map, lesion information associated with the target breast, the lesion information comprising lesion position information and benign-malignant status information; and adding an annotation associated with the lesion information to a target scanning image acquired during the target scan.

A method for breast imaging is provided. The method is implemented on a computing device comprising at least one processor and at least one storage device. The method comprises: controlling a breast scanning device to compress a target breast to a compressed state and utilizing a pressure sensor provided on the breast scanning device to acquire an initial pressure distribution map of the target breast in the compressed state; performing, based on the initial pressure distribution map, a verification on a current positioning of the target breast; and in response to determining that the current positioning does not pass the verification, issuing a prompt to adjust the current positioning; or, in response to determining that the current positioning passes the verification, controlling the breast scanning device to perform a target scan on the target breast.

In some embodiments, the performing a verification on a current positioning of the target breast comprises: determining, based on the initial pressure distribution map, whether a positioning type of the current positioning is the same as a positioning type of a target positioning corresponding to the target scan; in response to determining that the positioning type of the current positioning is the same as the positioning type of the target positioning, determining that the current positioning passes the verification; or in response to determining that the positioning type of the current positioning is not the same as the positioning type of the target positioning, determining that the current positioning does not pass the verification.

In some embodiments, the determining, based on the initial pressure distribution map, whether a positioning type of the current positioning is the same as a positioning type of a target positioning corresponding to the target scan comprises: determining the positioning type of the current positioning by analyzing a pressure value distribution in the initial pressure distribution map; comparing the positioning type of the current positioning with the positioning type of the target positioning to determine whether the positioning type of the current positioning is the same as the positioning type of the target positioning.

In some embodiments, the determining, based on the initial pressure distribution map, whether a positioning type of the current positioning is the same as a positioning type of a target positioning corresponding to the target scan comprises: inputting the initial pressure distribution map into a position type determination model to obtain the positioning type of the current positioning; and comparing the positioning type of the current positioning with the positioning type of the target positioning to determine whether the positioning type of the current positioning is the same as the positioning type of the target positioning.

In some embodiments, the determining, based on the initial pressure distribution map, whether a positioning type of the current positioning is the same as a positioning type of a target positioning corresponding to the target scan comprises: obtaining a reference pressure distribution map corresponding to the target positioning; obtaining a similarity degree between the initial pressure distribution map and the reference pressure distribution map; and determining, based on the similarity degree, whether the positioning type of the current positioning is the same as the positioning type of the target positioning.

In some embodiments, the in response to determining that the positioning type of the current positioning is the same as the positioning type of the target positioning, determining that the current positioning passes the verification further comprises: in response to determining that the positioning type of the current positioning is the same as the positioning type of the target positioning, determining, based on the initial pressure distribution map, a relative position between a lesion tissue of the target breast and a thoracic wall side; determining, based on the relative position, whether the position of the lesion tissue needs to be adjusted; in response to determining that the position of the lesion tissue does not need to be adjusted, determining that the current positioning passes the verification.

In some embodiments, the breast scanning device comprises a localized compression paddle and a support table, the pressure sensor is disposed on a surface of the support table that is in contact with the target breast, the performing a verification on a current positioning of the target breast comprises: determining, based on the initial pressure distribution map, a relative position between a lesion tissue of the target breast and the localized compression paddle; determining, based on the relative position, whether the position of the lesion tissue needs to be adjusted; and in response to determining that the position of the lesion tissue does not need to be adjusted, determining that the current positioning passes the verification; or in response to determining that the position of the lesion tissue needs to be adjusted, determining that the current positioning does not pass the verification.

In some embodiments, in response to determining that the current positioning passes the verification, controlling the breast scanning device to perform a target scan on the target breast comprises: designating the initial pressure distribution map as a pressure distribution map and obtaining a reference identification result by identifying one or more target tissues of the target breast in the pressure distribution map, the one or more target tissues comprising at least one of a lesion tissue or a glandular tissue; determining, based on the reference identification result, one or more target scanning parameters; and controlling, based on the one or more target scanning parameters, the breast scanning device to perform the target scan on the target breast.

In some embodiments, in response to determining that the current positioning passes the verification, the method further comprises: obtaining a dynamic pressure distribution map, the dynamic pressure distribution map being acquired by the pressure sensor during a process of compressing the target breast to the compressed state; determining, based on the dynamic pressure distribution map, lesion information associated with the target breast, the lesion information comprising lesion position information and benign-malignant status information; adding an annotation associated with the lesion information to a target scanning image acquired by the target scan.

A method for breast imaging is provided. The method is implemented on a computing device comprising at least one processor and at least one storage device. The method comprises: controlling a breast scanning device to compress a target breast and utilizing a pressure sensor provided on the breast scanning device to acquire a dynamic pressure distribution map of the target breast during a compression process; controlling the breast scanning device to perform a target scan on the target breast after the compression process to acquire a target scanning image; determining, based on the dynamic pressure distribution map, lesion information associated with the target breast, the lesion information comprising lesion position information and benign-malignant status information; and processing, based on the lesion information, the target scanning image to generate a processed target scanning image.

In some embodiments, the processing the target scanning image comprises: determining target pressure distribution map corresponding to the target scanning image from the dynamic pressure distribution map, a compressed state of the target breast in the target scanning image being consistent with a compressed state of the target breast in the target pressure distribution map; determining, based on the target pressure distribution map, a position of a lesion tissue in the target scanning image; and adding an annotation at the position of the lesion tissue in the target scanning image.

In some embodiments, the method further comprises controlling a terminal device to simultaneously display the target pressure distribution map and the target scanning image with the annotation.

In some embodiments, the method further comprises fusing the target pressure distribution map and the target scanning image with the annotation to obtain a dual-modality fused image; and controlling a terminal device to display the dual-modality fused image.

In some embodiments, the dynamic pressure distribution map comprises a plurality of pressure distribution maps, the benign-malignant status information is determined by: for each pressure distribution map, identifying a lesion tissue in the pressure distribution map to determine a reference lesion region in the pressure distribution map; determining, based on the reference lesion regions in the plurality of pressure distribution maps, lesion change information; and determining, based on the lesion change information, the benign-malignant status information.

In some embodiments, the lesion change information comprises at least one of first change information of a lesion area over time or second change information of a lesion pressure over time.

In some embodiments, the dynamic pressure distribution map comprises a plurality of pressure distribution maps, the lesion position information further comprises lesion depth information, and the lesion depth information is determined by: for each pressure distribution map, identifying a lesion tissue in the pressure distribution map to determine a reference lesion region in each pressure distribution map; determining, based on the reference lesion regions in the plurality of pressure distribution maps, a lesion appearance time; determining, based on the lesion appearance time, the lesion depth information.

In some embodiments, before performing the target scan, the method further comprises: performing, based on the dynamic pressure distribution map, a verification on a current positioning of the target breast; and in response to determining that the current positioning passes the verification, controlling the breast scanning device to perform the target scan on the target breast; or in response to determining that the current positioning does not pass the verification, issuing a prompt to adjust the current positioning.

In some embodiments, the controlling the breast scanning device to perform a target scan on the target breast comprises: determining, based on the dynamic pressure distribution map, one or more target scanning parameters; controlling, based on the one or more target scanning parameters, the breast scanning device to perform the target scan on the target breast.

A system is provided. The system comprises at least one storage device storing a set of instructions for breast imaging and at least one processor configured to communicate with the at least one storage device, wherein when executing the set of instructions, the at least one processor is configured to direct the system to perform methods for breast imaging disclosed herein.

A non-transitory computer readable medium is provided. The non-transitory computer readable medium comprises a set of instructions for breast imaging, wherein when executed by at least one processor, the set of instructions direct the at least one processor to effectuate the methods for breast imaging disclosed herein.

### BRIEF DESCRIPTION OF THE DRAWINGS

The present disclosure is further described in detail by way of exemplary embodiments. These exemplary embodiments are described in detail with reference to the accompanying drawings. These embodiments are non-limiting. In these embodiments, the same reference numerals denote the same structures, wherein:

FIG. 1 is a schematic diagram illustrating a breast imaging system according to some embodiments of the present disclosure;

FIG. 2 is a schematic diagram illustrating modules of a breast imaging system according to some embodiments of the present disclosure;

FIG. 3 is a flowchart illustrating a process of breast imaging according to some embodiments of the present disclosure;

FIG. 4 is a flowchart illustrating a process of breast imaging according to some embodiments of the present disclosure;

FIG. 5 is a flowchart illustrating a process of breast imaging according to some embodiments of the present disclosure;

FIGS. 6A to 6C are schematic diagrams illustrating a process of determining whether a positioning type of a current positioning of a target breast is the same as that of a target positioning corresponding to a target scan according to some embodiments of the present disclosure;

FIG. 7A is a schematic diagram illustrating a current positioning corresponding to a pressure distribution map according to some embodiments of the present disclosure;

FIG. 7B is a schematic diagram illustrating a reference pressure distribution map corresponding to a target positioning according to some embodiments of the present disclosure;

FIG. 8 is a flowchart illustrating a process of breast imaging according to some embodiments of the present disclosure;

FIG. 9 is a schematic diagram illustrating a process of determining one or more target regions in a pre-scanning image according to some embodiments of the present disclosure;

FIG. 10 is a flowchart illustrating a process of determining a glandular threshold according to some embodiments of the present disclosure;

FIG. 11 is a flowchart illustrating a process of determining a target exposure parameter according to some embodiments of the present disclosure;

FIG. 12 is a flowchart illustrating a process of performing a target scan on a target breast according to some embodiments of the present disclosure;

FIG. 13A is a flowchart illustrating a process of determining a lesion identification result of a target breast according to some embodiments of the present disclosure;

FIG. 13B is a schematic diagram illustrating a pressure distribution map according to some embodiments of the present disclosure;

FIG. 13C is a schematic diagram illustrating a segmentation result corresponding to a pressure distribution map according to some embodiments of the present disclosure;

FIG. 14 is a flowchart illustrating a process of performing a target scan on a target breast according to some embodiments of the present disclosure;

FIG. 15 is a flowchart illustrating a process of breast imaging according to some embodiments of the present disclosure;

FIG. 16A is a flowchart illustrating a process of displaying a target scanning image according to some embodiments of the present disclosure;

FIG. 16B is a schematic diagram illustrating a dynamic pressure distribution map according to some embodiments of the present disclosure;

FIG. 16C is a schematic diagram illustrating a target pressure distribution map and a target scanning image with an annotation according to some embodiments of the present disclosure;

FIG. 16D is a schematic diagram illustrating a dual-modality fused image according to some embodiments of the present disclosure;

FIG. 17 is a flowchart illustrating a process of determining lesion information associated with a target breast according to some embodiments of the present disclosure;

FIG. 18 is a schematic diagram illustrating a localized compression paddle according to some embodiments of the present disclosure; and

FIG. 19 is a flowchart illustrating a process of breast imaging according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the terms “system,” “engine,” “unit,” “module,” and/or “block” used herein are one method to distinguish different components, elements, parts, sections, or assemblies of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The terms “pixel” and “voxel” in the present disclosure are used interchangeably to refer to an element of an image.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In the present disclosure, a representation of a subject (e.g., an object, a patient, or a portion thereof) in an image may be referred to as “subject” for brevity. For instance, a representation of an organ, tissue (e.g., a heart, a liver, a lung), or an ROI in an image may be referred to as the organ, tissue, or ROI, for brevity. Further, an image including a representation of a subject, or a portion thereof, may be referred to as an image of the subject, or a portion thereof, or an image including the subject, or a portion thereof, for brevity. Still further, an operation performed on a representation of a subject, or a portion thereof, in an image may be referred to as an operation performed on the subject, or a portion thereof, for brevity. For instance, a segmentation of a portion of an image including a representation of an ROI from the image may be referred to as a segmentation of the ROI for brevity.

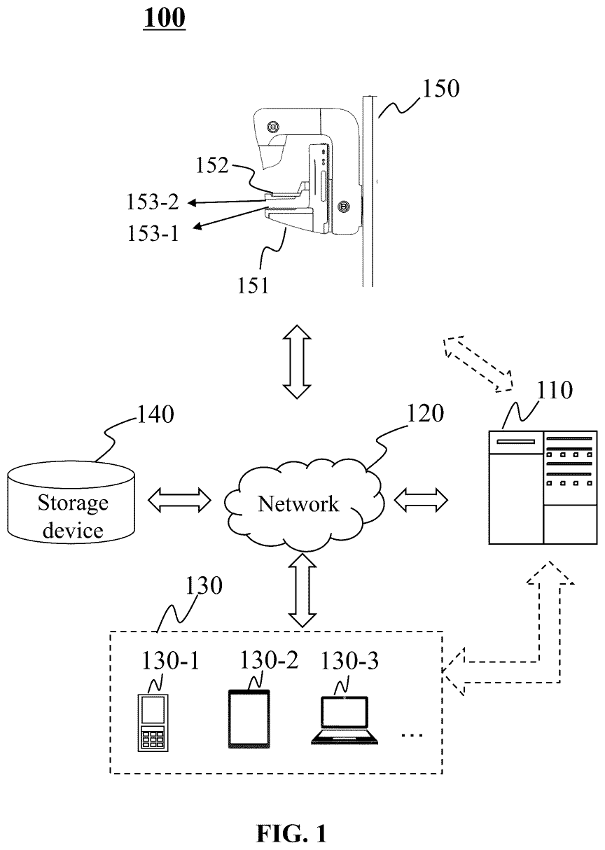

FIG. 1 is a schematic diagram illustrating a breast imaging system according to some embodiments of the present disclosure.

As shown in FIG. 1, a breast imaging system 100 includes a processing device 110, a network 120, a terminal device 130, a storage device 140, and a breast scanning device 150.

The processing device 110 may process data and/or information obtained from the terminal device 130, the storage device 140, and the breast scanning device 150. For example, the processing device 110 determines one or more target scanning parameters based on a pressure distribution map collected before a target scan, and controls the breast scanning device 150 to perform the target scan on a target breast based on the one or more target scanning parameters. As another example, the processing device 110 obtains a target scanning image from the breast scanning device 150 acquired during the target scan.

In some embodiments, the processing device 110 may be integrated into the breast scanning device 150. In some embodiments, the processing device 110 may be a single server or a server group. In some embodiments, the processing device 110 may be local or remote. The processing device 110 may be directly connected to the terminal device 130, the storage device 140, and the breast scanning device 150 to access stored or obtained information and/or data. In some embodiments, the processing device 110 may be implemented on a cloud platform. Merely by way of example, the cloud platform includes a private cloud, a public cloud, a hybrid cloud, a community cloud, a distributed cloud, an internal cloud, a multi-layer cloud, or the like, or any combination thereof. In some embodiments, the processing device 110 may be a distributed server group including a plurality of server nodes.

The network 120 may include any suitable network that facilitates information and/or data exchange of the breast imaging system 100. In some embodiments, one or more components of the breast imaging system 100 (e.g., the terminal device 130, the processing device 110, the storage device 140, or the breast scanning device 150) may communicate information and/or data with one or more other components of the breast imaging system 100 via the network 120. For example, the processing device 110 obtains a pressure distribution map and/or an exposure image (e.g., the target scanning image) from the breast scanning device 150 and/or the storage device 140 via the network 120.

In some embodiments, the network 120 may be any one or more of a wired network or a wireless network. In some embodiments, the network 120 may have various topologies such as point-to-point, shared, centralized, or any combination of multiple topologies.

The terminal device 130 may include a mobile device 130-1, a tablet computer 130-2, a laptop computer 130-3, or the like, or any combination thereof. In some embodiments, the terminal device 130 interacts with other components of the breast imaging system 100 via the network 120. In some embodiments, the terminal device 130 receives information and/or instructions input by a user, and sends the received information and/or instructions to the processing device 110 via the network 120. For example, the terminal device 130 receives an instruction from a user (e.g., a physician), obtains a target scanning image with an annotation from the processing device 110 via the network 120, and displays the target scanning image.

The storage device 140 may store data and/or instructions. In some embodiments, the storage device 140 may store data obtained from the processing device 110, the terminal device 130, and/or the breast scanning device 150. For example, the storage device 140 stores data (e.g., a pressure distribution map) acquired by the breast scanning device 150. In some embodiments, the storage device 140 may store data and/or instructions for the processing device 110 to execute exemplary methods described in the present disclosure. For example, the storage device 140 stores instructions for the processing device 110 to execute methods illustrated in various flowcharts. In some embodiments, the storage device 140 may include a mass storage device, a removable storage device, a volatile read-and-write memory, a read-only memory (ROM), or the like, or any combination thereof. In some embodiments, the storage device 140 may be implemented on a cloud platform. In some embodiments, the storage device 140 may be part of the processing device 110.

The breast scanning device 150 refers to a medical device that can be used for imaging a target breast of a patient. The breast scanning device 150 may include a breast X-ray imaging device, a digital breast tomosynthesis (DBT) device, full-field digital mammography (FFDM), contrast-enhanced digital mammography (CEDM), contrast-enhanced digital breast tomosynthesis (CEDBT), or the like.

In some embodiments, the breast scanning device 150 includes a breast compression device configured to compress the target breast of the patient. As shown in FIG. 1, the breast compression device includes a support table 151 and a compression paddle 152. The support table 151 may be configured to place the target breast of the patient (e.g., a left breast or a right breast). The compression paddle 152 is arranged opposite to the support table 151 and may be configured to compress (or squeeze) the target breast, thereby causing deformation of the target breast.

The breast scanning device 150 may control the movement (e.g., translation) of the compression paddle 152 toward the support table 151 based on different compression force parameters (e.g., 5N, 10N), thereby reducing a distance between the compression paddle 152 (a lower surface of the compression paddle 152) and the support table 151 (an upper surface of the support table 151). In this way, the target breast placed on the upper surface of the support table 151 is compressed. The distance may be referred to as a compression thickness. When a preset compression thickness is reached, the compression paddle 152 stops moving, and the target breast remains in a stable compressed state.

The target breast may be compressed when the target breast is under a preset positioning. Such positioning may be achieved by requiring the patient to adjust a posture of the target breast, or by changing a rotation angle of the breast compression device. Merely by way of example, the patient places the target breast on the upper surface of the support table 151 while standing in a vertical posture. The breast scanning device 150 controls the rotation of the breast compression device to apply a compression to different sides of the target breast (e.g., the left side, the right side, or the like).

The breast scanning device 150 may further include a pressure sensor configured to acquire pressure information of the target breast during and/or after the compression. The pressure information may include a pressure force value, a pressure intensity value, or the like. In some embodiments, the pressure sensor includes a thin-film pressure sensor. As shown in FIG. 1, the pressure sensor includes a thin-film pressure sensor 153-1 disposed on the upper surface of the support table 151 and/or a thin-film pressure sensor 153-2 disposed on the lower surface of the compression paddle 152. The upper surface of the support table 151 and the lower surface of the compression paddle 152 refer to surfaces that contact (or fit) the target breast. In some embodiments, the pressure sensor includes a sensor array including a plurality of pressure-sensing units. In some embodiments, the pressure sensor may include one or more of the following types: a strain-gauge pressure sensor, a piezoresistive pressure sensor, a piezoelectric pressure sensor, a resonant pressure sensor, a thin-film pressure sensor, a micro-electro-mechanical system (MEMS) pressure sensor, or the like.

The pressure information acquired by the pressure sensor at a certain time may be represented by a pressure distribution map. The pressure distribution map includes values (e.g., pressure values or pressure intensity values) corresponding to different regions of the target breast (e.g., an adipose region, a glandular region, a lesion region) at that time. The pressure information acquired by the pressure sensor over a period of time may be represented by a dynamic pressure distribution map. For example, the dynamic pressure distribution map reflects a change in pressure values of the target breast during a compression process (from the initiation of the compression on the target breast until the target breast is in the compressed state). The dynamic pressure distribution map may include pressure distribution maps corresponding to a plurality of times during the compression process.

In some embodiments, each pixel point in the pressure distribution map corresponds to a physical point on the target breast. A size of each pixel point may be set according to actual needs. Merely by way of example, the pixel points may be square cells of size a×a, where a is a preset value, and each square cell may be regarded as a subregion in the pressure distribution map. A pressure value of each physical point may be represented by a grayscale value of a corresponding pixel point. The pressure value of each physical point may also be represented by a color of a corresponding pixel point. Merely by way of example, when a pressure value of a physical point is within a first pressure range, a corresponding pixel point is represented in red. When a pressure value of a physical point is within a second pressure range, a corresponding pixel point is represented in yellow. When a pressure value of a physical point is within a third pressure range, a corresponding pixel point is represented in blue.

The pressure distribution map may reflect a distribution of different breast tissues (e.g., an adipose tissue, a glandular tissue, a lesion tissue, or the like) in the target breast. It may be understood that different breast tissues have different hardness/density. During the compression process in which the compression paddle applies a compression based on the same compression parameter (e.g., a compression thickness, a compression force), pressure values received by the pressure sensor are different. Merely by way of example, the hardness of an adipose tissue, a glandular tissue, a benign mass, and a malignant mass increases sequentially. Correspondingly, the pressure values corresponding to the adipose tissue, the glandular tissue, the benign mass, and the malignant mass increase, and the pixel values of corresponding pixel points in a pressure distribution map also increase.

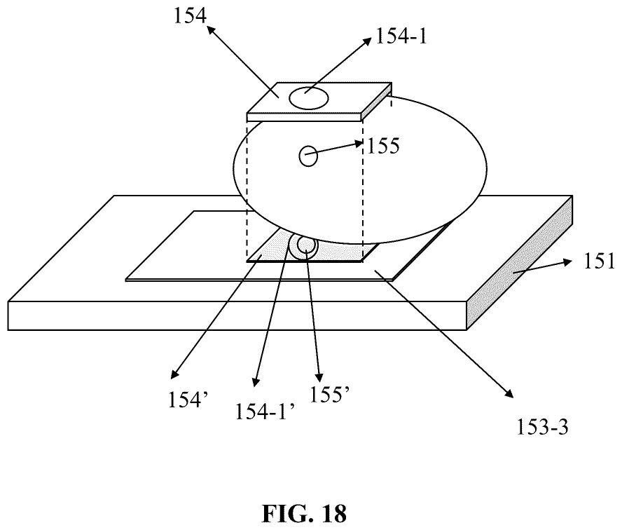

In some embodiments, the compression paddle 152 may be a localized compression paddle configured to compress a local tissue (e.g., a lesion tissue) of the target breast. FIG. 18 is a schematic diagram illustrating a localized compression paddle according to some embodiments of the present disclosure. As shown in FIG. 18, the breast scanning device 150 includes a localized compression paddle 154. A puncture hole 154-1 is opened on the localized compression paddle 154. A puncture needle may be inserted via the puncture hole 154-1 to puncture a lesion tissue 155 of the target breast. A pressure sensor 153-3 is disposed on a surface of the support table 151 that contacts the target breast. A pressure distribution map acquired by the pressure sensor 153-3 may be used to determine whether a position of the lesion tissue is within a range corresponding to the puncture hole 154-1.

In some embodiments, the breast scanning device 150 includes components such as a radiation source and a detector configured to acquire a scanning image of the target breast. Merely by way of example, the breast scanning device 150 performs a scan on the target breast in a compressed state based on one or more target scanning parameters (e.g., a target scanning angle, a target exposure parameter) to acquire a target scanning image of the target breast.

The breast imaging system 100 may further comprise other components. For example, the breast imaging system 100 further comprises a voice acquisition device configured to acquire voice feedback information from a patient.

The above description is for illustrative purposes only. Actual application scenarios may have various changes. It should be noted that the breast imaging system 100 is provided only for illustrative purposes and is not intended to limit the scope of the present disclosure. For those of ordinary skill in the art, various modifications or changes may be made according to the description of the present disclosure. However, these changes and modifications do not depart from the scope of the present disclosure.

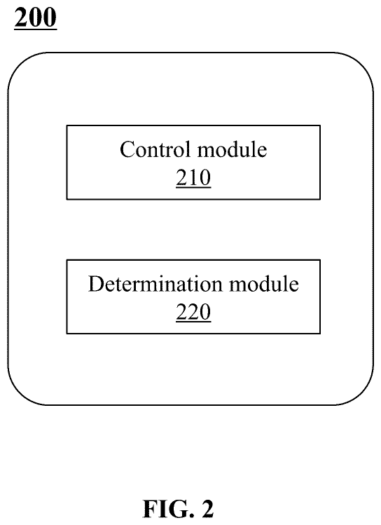

FIG. 2 is a schematic diagram illustrating modules of a breast imaging system according to some embodiments of the present disclosure. As shown in FIG. 2, a breast imaging system 200 may include a control module 210 and a determination module 220.

The control module 210 may be configured to control a breast scanning device and/or a pressure sensor. For example, the control module 210 may control the breast scanning device to compress a target breast based on a compression parameter (e.g., initial compression parameter, a target compression parameter), and may control the pressure sensor to acquire a pressure distribution map of the target breast (e.g., an initial pressure distribution map, a pressure distribution map, a dynamic pressure distribution map, etc.).

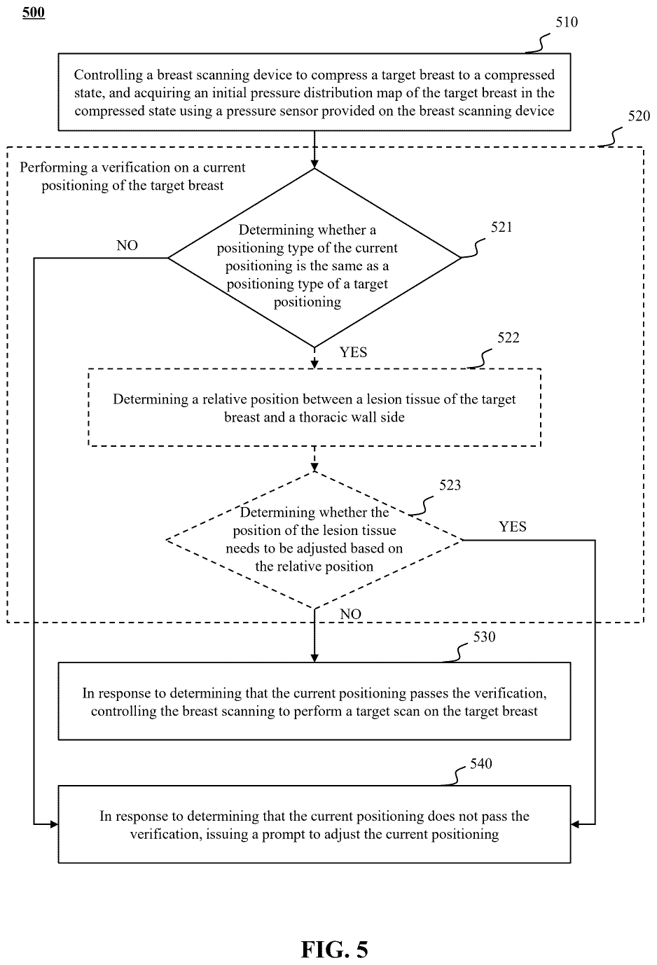

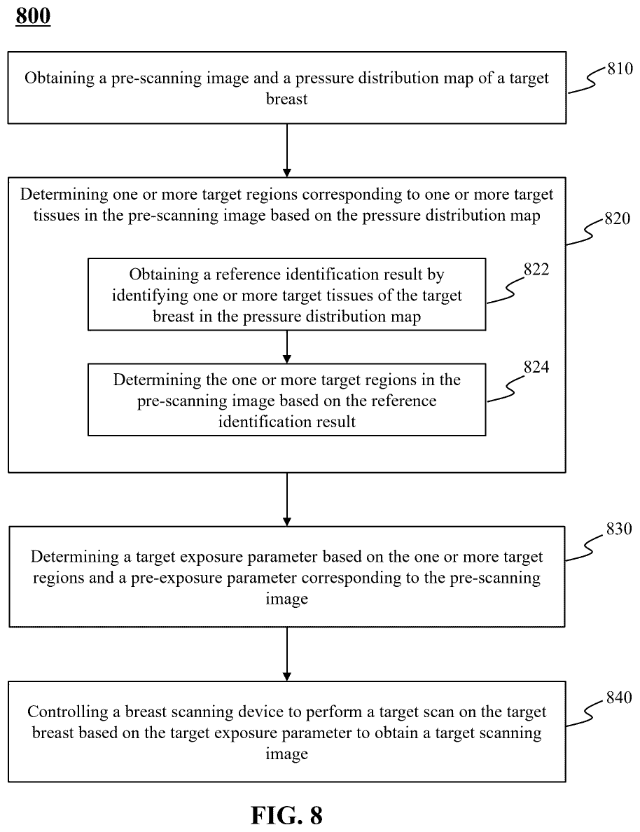

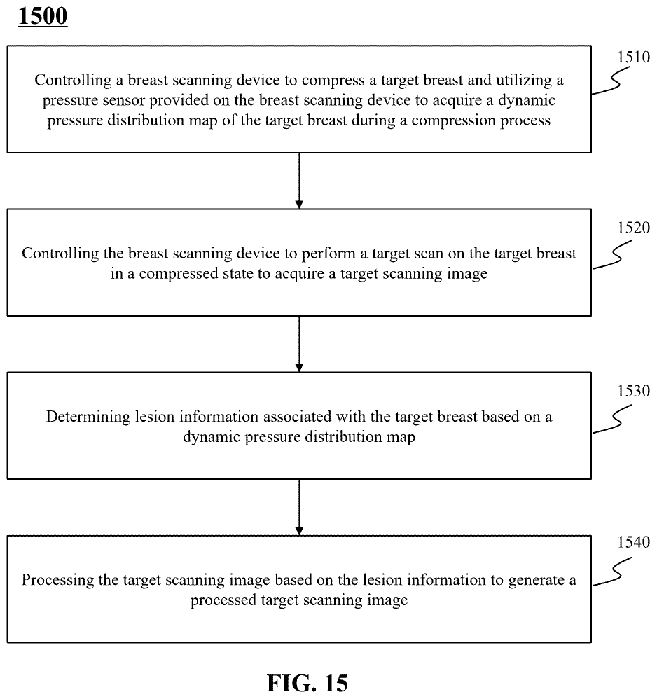

The determination module 220 may be configured to determine whether a current positioning passes a verification, determine one or more target scanning parameters, and generate a processed target scanning image. Detailed descriptions of determining whether a current positioning passes a verification may be found in the related descriptions of FIG. 5 to FIG. 7. Detailed descriptions of determining one or more target scanning parameters may be found in the related descriptions of FIG. 8 to FIG. 14. Detailed descriptions of generating a processed target scanning image may be found in the related descriptions of FIG. 15 to FIG. 17.

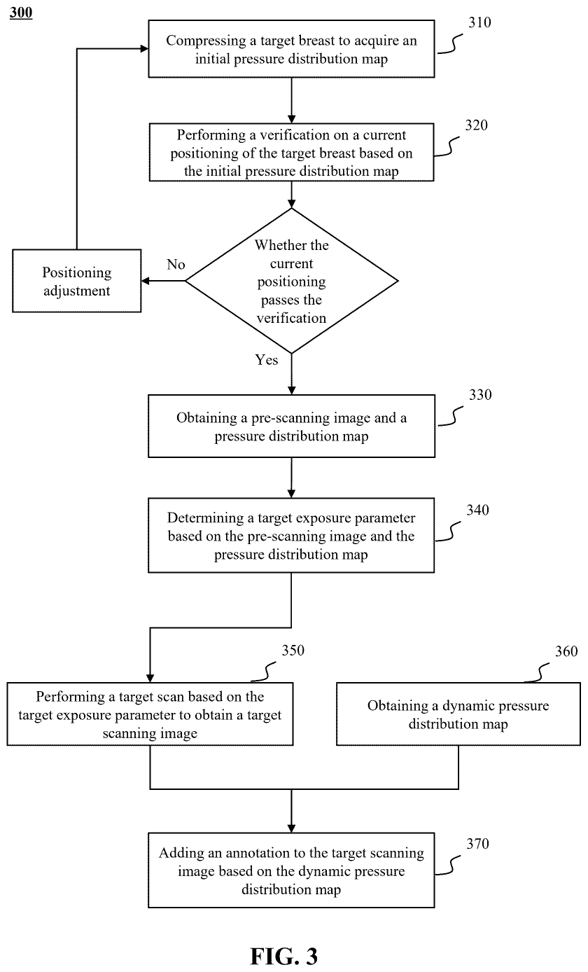

FIG. 3 is a flowchart illustrating a process of breast imaging according to some embodiments of the present disclosure. In some embodiments, a process 300 may be performed by a breast imaging system (e.g., the processing device 110). As shown in FIG. 3, the process 300 includes operations 310-370. Operations 310-340 are executed before performing a target scan and are used to prepare for the target scan.

In operation 310, a target breast may be compressed to acquire an initial pressure distribution map.

The initial pressure distribution map refers to a pressure distribution map used to perform a positioning verification. In some embodiments, the processing device 110 controls a breast scanning device to compress the target breast based on an initial compression parameter to acquire the initial pressure distribution map of the target breast. In some embodiments, if a target compression parameter for the target scan is pre-determined, the processing device 110 may control the breast scanning device to compress the target breast based on the target compression parameter to acquire the initial pressure distribution map of the target breast. Operation 310 is performed in a similar manner to operation 510 described hereinafter.

In operation 320, a verification may be performed on a current positioning of the target breast based on the initial pressure distribution map.

The processing device 110 may perform the verification on the current positioning of the target breast based on the initial pressure distribution map. Merely by way of example, whether a positioning type of the current positioning of the target breast is the same as a positioning type of a target positioning corresponding to the target scan is determined based on the initial pressure distribution map. In response to determining that the positioning type of the current positioning is the same as the positioning type of the target positioning corresponding to the target scan, it is determined that the current positioning passes the verification. Then the processing device 110 executes operation 330. Or, in response to determining that the positioning type of the current positioning is not the same as the positioning type of the target positioning corresponding to the target scan, it is determined that the current positioning does not pass the verification. Then the processing device returns to execute operation 310 and operation 320.

In some embodiments, in response to determining that the current positioning does not pass the verification, the processing device 110 issues a prompt to assist the patient in adjusting the current positioning or to remind a physician to perform a positioning adjustment for the patient. After the positioning adjustment, the processing device 110 executes operation 310 and operation 320 again until the current positioning passes the verification. More descriptions related to the positioning verification may be found in FIG. 5 and the related descriptions thereof.

In operation 330, a pre-scanning image and a pressure distribution map may be obtained.

The processing device 110 may control the breast imaging device to perform a pre-exposure on the target breast based on a pre-exposure parameter to obtain the pre-scanning image. At the same time, the processing device 110 may obtain a pressure distribution map acquired by the pressure sensor during the pre-exposure. The pressure distribution map and the pre-scanning image are acquired simultaneously, and the pressure distribution map may reflect a pressure distribution of the target breast in the compressed state when acquiring the pre-scanning image. More descriptions related to the pre-scanning image and the pressure distribution map may be found in FIG. 8 and the related descriptions thereof.

In some embodiments, in response to determining that the current positioning passes the verification based on the initial pressure distribution map in operation 310, the initial pressure distribution map may be designated as the pressure distribution map. That is, the initial pressure distribution map and the pressure distribution map are the same image.

In operation 340, a target exposure parameter may be determined based on the pre-scanning image and the pressure distribution map.

In some embodiments, the processing device 110 may determine a target region in the pre-scanning image based on the pressure distribution map, and determine the target exposure parameter based on the target region and the pre-exposure parameter corresponding to the pre-scanning image. More descriptions may be found in FIG. 8 and the descriptions thereof.

In operation 350, a target scan may be performed based on the target exposure parameter to obtain a target scanning image.

In some embodiments, the processing device 110 may control the breast scanning device to perform the target scan on the target breast based on the target exposure parameter to obtain the target scanning image. The target breast is in the compressed state in the target scan.

In operation 360, a dynamic pressure distribution map may be obtained.

The dynamic pressure distribution map may reflect a change in a pressure value distribution of the target breast over time throughout the compression process. A compressed state of the target breast in the initial pressure distribution map and a compressed state of the target breast in the pressure distribution map may be the same or different. For example, the target scan is performed based on the target compression parameter. If the target breast is compressed based on the target compression parameter corresponding to the target scan in operation 310, no compression adjustment on the target breast is required in operation 330, and the dynamic pressure distribution map may be acquired during a process of compressing the target breast while executing operation 310. If the target breast is compressed based on the initial compression parameter in operation 310, the target breast needs to be re-compressed based on the target compression parameter in operation 330, and the dynamic pressure distribution map may be acquired during a process of compressing the target breast based on the target compression parameter. More descriptions of the dynamic pressure distribution map may be found in other parts of the present disclosure (e.g., FIG. 15).

In operation 370, an annotation may be added to the target scanning image based on the dynamic pressure distribution map.

The processing device 110 may analyze the dynamic pressure distribution map to determine lesion information associated with the target breast. The lesion information includes benign-malignant status information, lesion position information (e.g., a planar position, a depth), or the like.

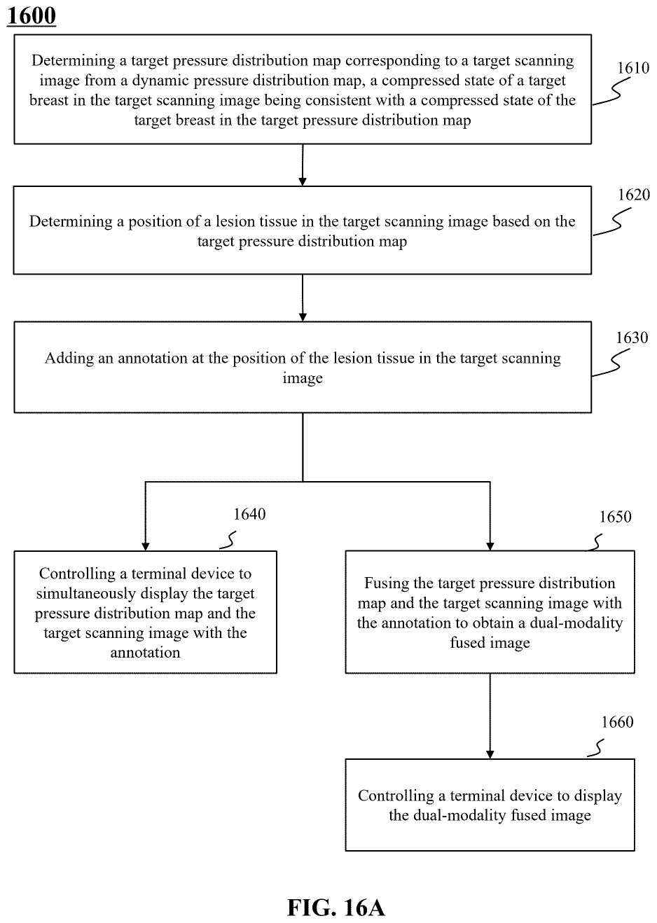

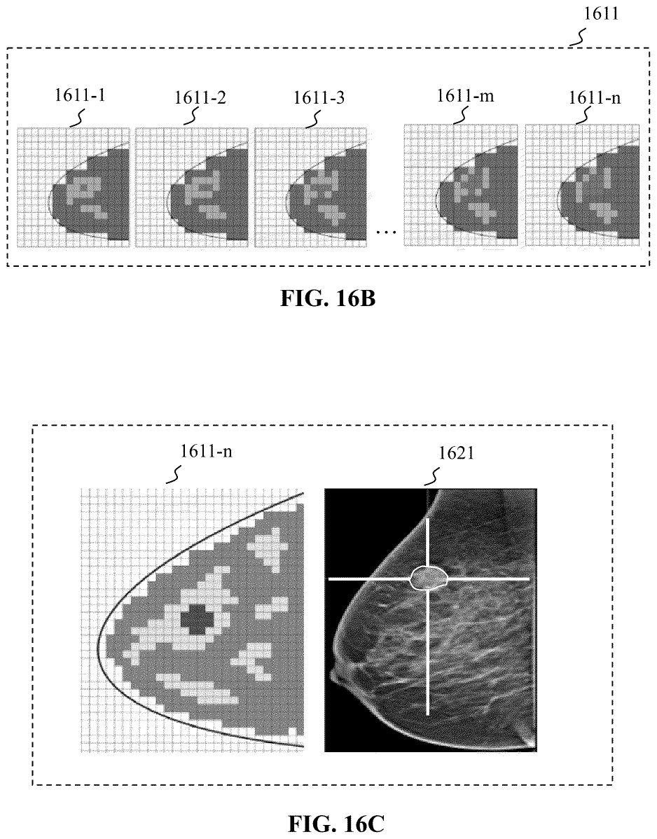

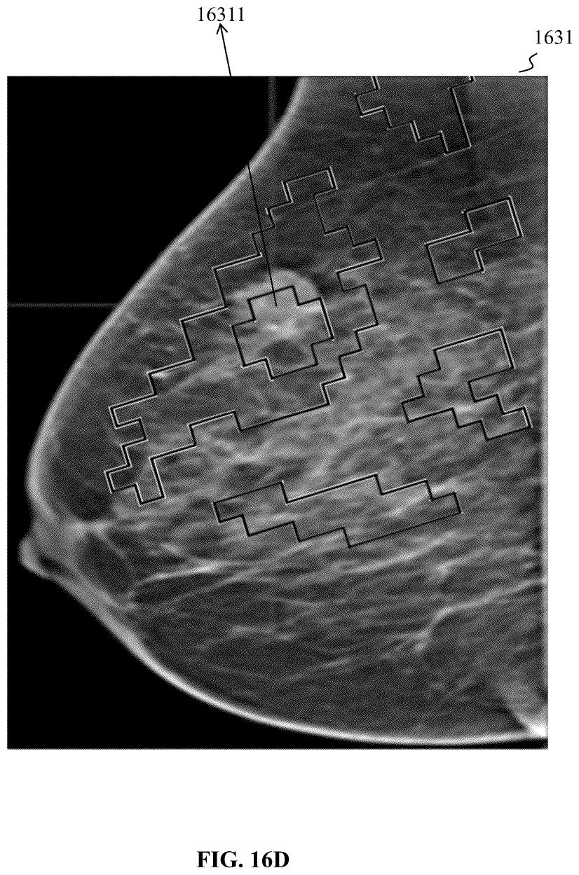

The annotation added to the target scanning image may be determined based on the lesion information. For example, the annotation indicates a position (e.g., a contour) of the lesion, a status of the lesion, or the like. The target scanning image with the annotation may be used for display on a terminal device (e.g., the terminal device 130) to assist a user (e.g., a physician) in image interpretation and/or diagnostic analysis. More descriptions of the target scanning image and the annotation may be found in other parts of the present disclosure (e.g., FIG. 15 and FIG. 16A).

In some embodiments of the present disclosure, by acquiring the pressure distribution map and the pre-scanning image during a preparation stage, a more accurate exposure parameter can be obtained, reducing the acquisition of invalid images during a scanning stage and minimizing unnecessary radiation exposure to the patient or physician.

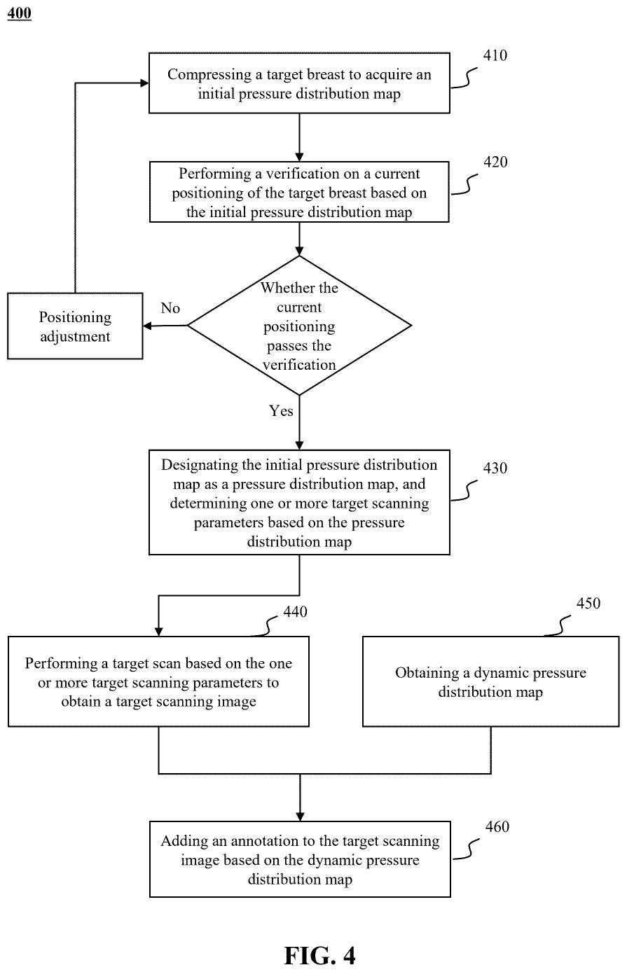

FIG. 4 is a flowchart illustrating a process of breast imaging according to some embodiments of the present disclosure; In some embodiments, a process 400 may be performed by the processing device 110. As shown in FIG. 4, the process 400 includes the following operations.

In operation 410, a target breast may be compressed to acquire an initial pressure distribution map.

Operation 410 may be performed in a similar manner to operation 310, and the descriptions thereof are not repeated here.

In operation 420, a verification may be performed on a current positioning of the target breast based on the initial pressure distribution map.

In operation 420 is performed in a similar manner to operation 320, and the descriptions thereof are not repeated here.

In response to determining that the current positioning passes the verification, operation 430 may be executed. In response to determining that the current positioning does not pass the verification, a positioning adjustment may be performed, and the process returns to operation 410 and operation 420.

In operation 430, the initial pressure distribution map may be designated as a pressure distribution map, and one or more target scanning parameters may be determined based on the pressure distribution map.

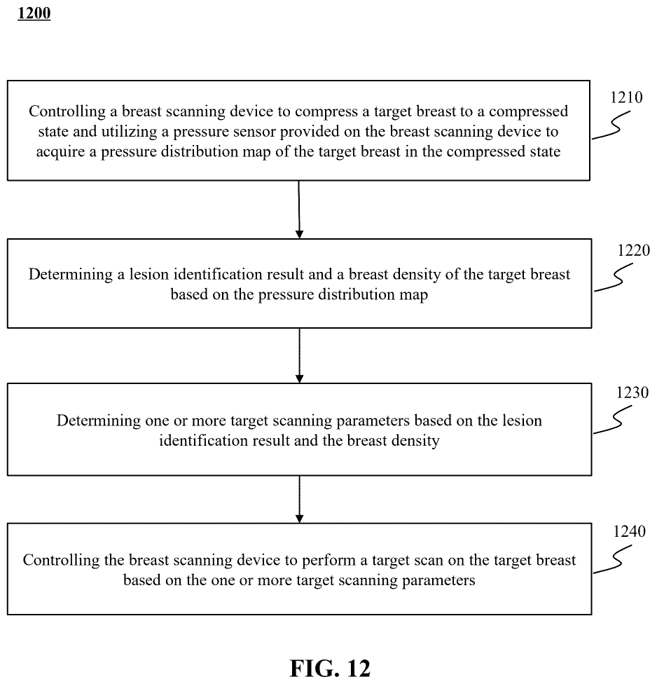

The processing device 110 may determine a lesion identification result and a breast density of the target breast based on the pressure distribution map, and determine the one or more target scanning parameters based on the lesion identification result and the breast density. The one or more target scanning parameters may include at least one of a target compression parameter, a target scanning angle, or a target exposure parameter. Merely by way of example, a smaller breast density corresponds to a smaller target scanning angle. More descriptions of determining the one or more target scanning parameters based on the pressure distribution map may be found in other parts of the present disclosure (e.g., FIG. 12).

In operation 440, a target scan may be performed based on the one or more target scanning parameters to obtain a target scanning image.

In response to determining that the target compression parameter is different from a compression parameter used in operation 410, the processing device 110 may control a breast scanning device to re-compress the target breast based on the target compression parameter, and control the breast scanning device to perform the target scan on the target breast based on the target scanning angle and the target exposure parameter. In response to determining that the target compression parameter is the same as the compression parameter used in operation 410, the processing device 110 may directly control the breast scanning device to perform the target scan on the target breast based on the target scanning angle and the target exposure parameter. More descriptions may be found in FIG. 12 and the descriptions thereof.

In operation 450, a dynamic pressure distribution map may be obtained.

The dynamic pressure distribution map in operation 450 refers to a dynamic pressure distribution map acquired by a pressure sensor throughout a process in which the breast scanning device compresses the target breast based on the target compression parameter. Operation 450 may be performed in a similar manner to operation 360, and the descriptions thereof are not repeated here.

In operation 460, an annotation may be added to the target scanning image based on the dynamic pressure distribution map.

Operation 460 may be performed in a similar manner as operation 370, and the descriptions thereof are not repeated here.

In some embodiments of the present disclosure, by acquiring the initial pressure distribution map during a preparation stage to verify the current positioning and determine the one or more target scanning parameters, unnecessary pre-exposure can be avoided, thereby reducing radiation exposure to the patient. At the same time, scanning parameters can be automatically and accurately determined, reducing excessive reliance on users (e.g., a physician) and minimizing the workload during the preparation stage.

FIG. 5 is a flowchart illustrating a process of breast imaging according to some embodiments of the present disclosure. In some embodiments, the processing device 110 or the breast imaging system 200 may execute a process 500. As shown in FIG. 5, the process 500 may include the following operations.

In operation 510, a breast scanning device may be controlled to compress a target breast to a compressed state, and an initial pressure distribution map of the target breast in the compressed state may be acquired by a pressure sensor provided on the breast scanning device.

The target breast refers to a breast of a target subject (e.g., a patient) that requires medical diagnosis (e.g., tumor detection, treatment), such as a left breast or a right breast of the patient. The target breast includes glandular tissues and adipose tissues, and may also include benign lesions and/or malignant lesions.

The initial pressure distribution map refers to a pressure distribution map of the target breast in the compressed state acquired by the pressure sensor before a target scan, which is used for positioning verification and/or scanning parameter determination. The compressed state refers to a state of the target breast after being compressed based on a specific compression parameter. In some embodiments, the pressure sensor is provided on a surface of a compression paddle and/or a support table of the breast scanning device that contacts the target breast.

In some embodiments, the processing device 110 controls the compression paddle to compress the target breast based on an initial compression parameter. Merely by way of example, the initial compression parameter includes an initial compression thickness and an initial compression force. After the compression is completed, a pressure distribution map acquired by the pressure sensor may be designated as the initial pressure distribution map. The compression on the target breast based on the initial compression parameter before the target scan may also be referred to as a pre-compression, and the compressed state of the target breast at this time may also be referred to as an initial compressed state. The pre-compression is used to acquire the initial pressure distribution map as preparation before the target scan (e.g., performing a positioning verification, determining one or more target scanning parameters).

The initial compression parameter may be a system default parameter for breast imaging or may be set by a user. Alternatively, the initial compression parameter may be determined based on patient information (e.g., age, height, breast size, physique, or the like). In some embodiments, the initial compression parameter may be determined based on historical medical records of the patient. Merely by way of example, the initial compression parameter may be determined based on a compression thickness and/or a compression force corresponding to a most recent diagnostic medical record of the patient. Considering the patient information and/or the historical medical records of the patient, the initial compression parameter may be determined more quickly, thereby improving the efficiency of scan preparation.

In some embodiments, if a target compression parameter corresponding to the target scan is pre-determined, the processing device 110 controls the compression paddle to compress the target breast based on the target compression parameter, and designates a pressure distribution map acquired by the pressure sensor after the compression is completed as the initial pressure distribution map.

In operation 520, a verification may be performed on a current positioning of the target breast based on the initial pressure distribution map.

The current positioning of the target breast refers to a positioning of the target breast at an acquisition time of the initial pressure distribution map.

In some embodiments, as shown in FIG. 5, operation 520 includes operation 521.

In operation 521, based on the initial pressure distribution map, whether a positioning type of the current positioning is the same as a positioning type of a target positioning corresponding to the target scan may be determined. The target positioning corresponding to the target scan refers to a positioning that the target breast needs to maintain during the target scan. Exemplary positioning types include craniocaudal (CC), oblique, auxiliary positions (e.g., lateral oblique), or the like. Merely by way of example, when a vertical cross-sectional area or a lateral area of the target breast needs to be scanned, the positioning type of the target positioning is craniocaudal (CC) or medial-lateral oblique (MLO). For convenience of description, the positioning type of the current positioning and the positioning type of the target positioning are hereinafter referred to as a current positioning type and a target positioning type, respectively.

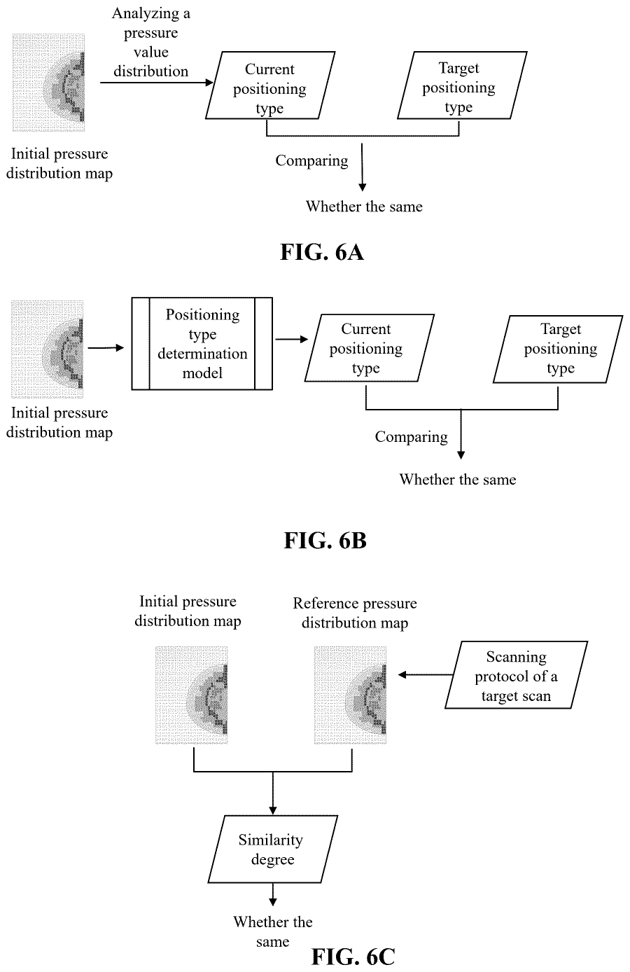

FIGS. 6A to 6C are schematic diagrams illustrating a process of determining whether a positioning type of a current positioning of a target breast is the same as that of a target positioning corresponding to a target scan according to embodiments of the present disclosure.

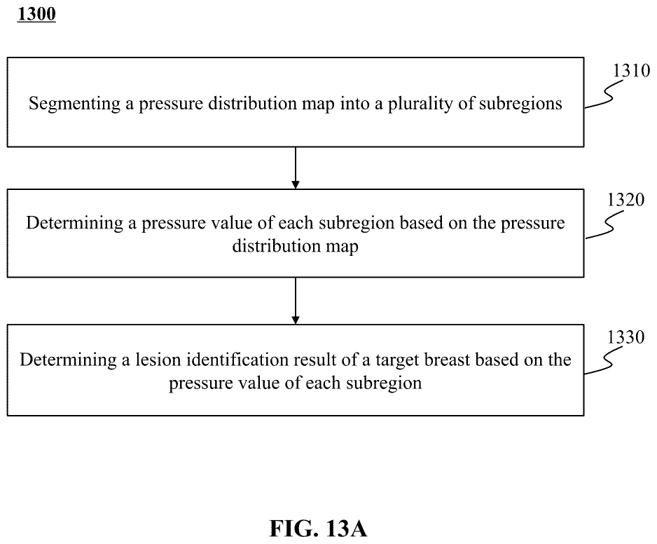

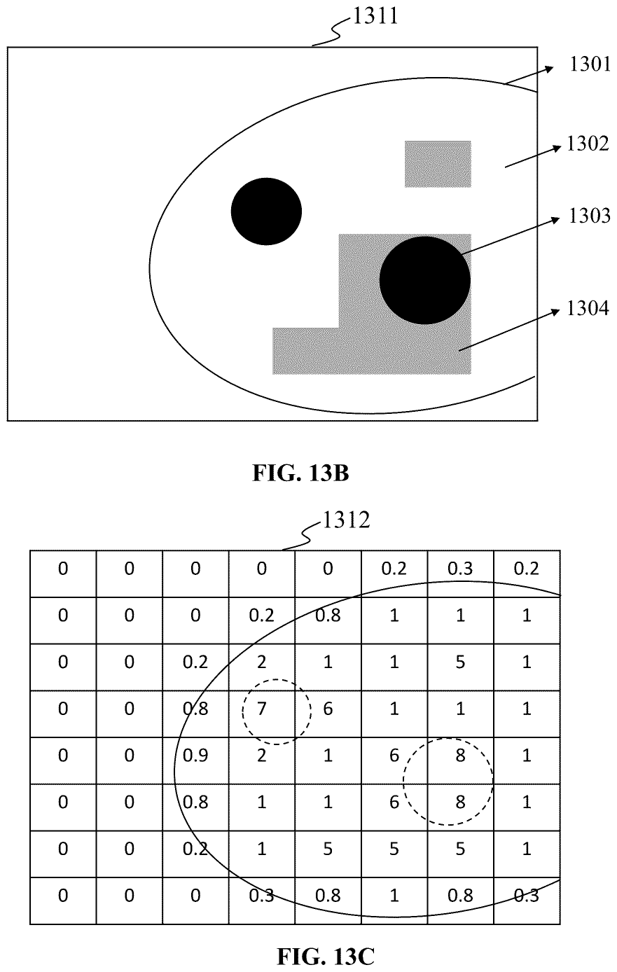

As shown in FIG. 6A, the processing device 110 may determine a current positioning type by analyzing the pressure value distribution in the initial pressure distribution map. For example, the processing device 110 may segment the initial pressure distribution map into a plurality of subregions, determine regions among the plurality of subregions where a pressure value (e.g., an average pressure value) is greater than 0, and determine the current positioning type based on the pressure value distribution in the regions. For example, the pressure value distribution in the regions is analyzed to determine a lesion region, a glandular region, and an adipose region, and the current positioning type may be determined based on corresponding positions of the lesion region, the glandular region, and the adipose region in the initial pressure distribution map. More descriptions regarding the segmentation of the subregions and the determination of different regions may be found in the related description of FIGS. 13A-13C.

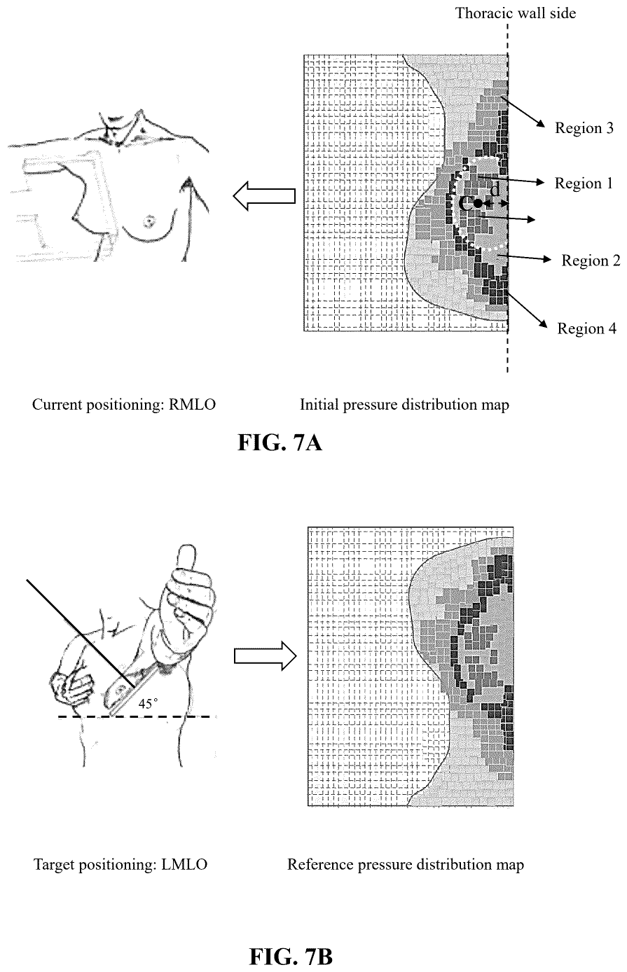

Merely by way of example, FIG. 7A is a schematic diagram illustrating a current positioning corresponding to an initial pressure distribution map according to some embodiments of the present disclosure. As shown in FIG. 7A, the processing device 110 may determine that the current positioning type is right medial-lateral oblique (RMLO) based on an outer contour and/or a morphology of regions with pressure values in the initial pressure distribution map.

Further, the processing device 110 may compare the current positioning type with the target positioning type to determine whether the current positioning type of the target breast is the same as the target positioning type. For example, continuing the above example, if the target positioning type is RMLO, the current positioning type of the target breast is the same as the target positioning type.

As shown in FIG. 6B, the processing device 110 may input an initial pressure distribution map into a positioning type determination model to obtain a current positioning type output by the positioning type determination model.

The positioning type determination model refers to a model for determining a positioning type of a breast in a pressure distribution map. In some embodiments, the positioning type determination model may include a convolutional neural network (CNN) model, a deep neural network (DNN), or the like. In some embodiments, the positioning type determination model may be obtained by training an initial model using a sample training set. The training sample set may include a plurality of first training samples and a plurality of first training labels corresponding to the plurality of first training samples. The first training sample may be a historical pressure distribution map acquired in a historical scan. The first training label may be a positioning type corresponding to the historical pressure distribution map. The first training label may be determined manually by a user.

Further, the processing device 110 may compare the current positioning type with the target positioning type to determine whether the current positioning type is the same as the target positioning type.

As shown in FIG. 6C, the processing device 110 may obtain a reference pressure distribution map corresponding to a target positioning. The reference pressure distribution map may reflect a pressure distribution of a target breast under the target positioning. In some embodiments, the processing device 110 may predict the reference pressure distribution map of the target breast under the target positioning based on a preset compression parameter through data simulation. Merely by way of example, the processing device 110 may establish a breast model based on historical scanning images of the target breast, and then perform data simulation based on the breast model to obtain the reference pressure distribution map. In some embodiments, the processing device 110 may obtain a pressure distribution map of a reference breast under the target positioning based on a preset compression parameter, and designate the pressure distribution map as the reference pressure distribution map. The reference breast refers to a breast that has subjected to positioning or scanning under the target positioning based on the preset compression parameter. FIG. 7B is a schematic diagram illustrating a reference pressure distribution map corresponding to a target positioning according to some embodiments of the present disclosure. As shown in FIG. 7B, when the target positioning type of the target positioning is left medial-lateral oblique (LMLO), the processing device 110 may obtain a reference pressure distribution map corresponding to the target positioning.

Further, the processing device 110 may obtain a similarity degree between the initial pressure distribution map and the reference pressure distribution map. The similarity degree between the initial pressure distribution map and the reference pressure distribution map refers to a parameter for evaluating a consistency degree between the initial pressure distribution map and the reference pressure distribution map. A greater similarity degree indicates a higher consistency between the initial pressure distribution map and the reference pressure distribution map. In some embodiments, the processing device 110 may determine the similarity degree between the initial pressure distribution map and the reference pressure distribution map using a pixel value comparison method, a feature point comparison method, a histogram comparison method, etc. In some embodiments, the processing device 110 may first convert the initial pressure distribution map and the reference pressure distribution map into binary images and then calculate a similarity degree between the binary images. This approach can avoid image differences caused by variations in the breast composition between the initial pressure distribution map and the reference pressure distribution map, allowing the similarity degree to more accurately reflect differences in positioning and preventing individual patient differences (e.g., variations in breast conditions) from affecting the accuracy of the assessment.

Furthermore, the processing device 110 may determine, based on the similarity degree, whether the current positioning type is the same as the target positioning type. In some embodiments, in response to determining that the similarity degree is greater than or equal to a similarity degree threshold, the processing device 110 determines that the current positioning type is the same as the target positioning type; or in response to determining that the similarity degree is less than the similarity degree threshold, the processing device 110 determines that the current positioning type is different from the target positioning type. The similarity degree threshold may be a system default value or manually set by a user.

In response to determining that the positioning type of the current positioning is different from the positioning type of the target positioning, the processing device 110 determines that the current positioning does not pass the verification, and executes operation 540.

In response to determining that the positioning type of the current positioning is the same as the positioning type of the target positioning, the processing device 110 determines that the current positioning passes the verification, and executes operation 530. In some embodiments, as shown in FIG. 5, in response to determining that the positioning type of the current positioning is the same as the positioning type of the target positioning, the processing device executes operation 522 and operation 523.

In operation 522, a relative position between a lesion tissue of the target breast and a thoracic wall side may be determined based on the initial pressure distribution map.

The lesion tissue refers to a tissue region where a lesion (e.g., a mass) exists. Merely by way of example, a lesion region encircled by the white dashed line in an arc-like shape in FIG. 7A indicates a lesion tissue. The thoracic wall side refers to a side of the pressure sensor that is closer to a standing position of the patient. Merely by way of example, when the patient stands on a right side of the pressure sensor, a right boundary of the initial pressure distribution map represents the thoracic wall side.

The relative position between the lesion tissue and the thoracic wall side is used to represent a positional relationship between the lesion tissue and the thoracic wall side, and may be represented by the shortest distance between a specific position of the lesion tissue (e.g., a center point or an edge point closest to the thoracic wall side) and the thoracic wall side. Merely by way of example, as shown in FIG. 7A, the relative position between the lesion tissue and the thoracic wall side may be represented by the shortest distance d between a center point C of the lesion tissue in the initial pressure distribution map and the thoracic wall side.

In operation 523, whether the position of the lesion tissue needs to be adjusted may be determined based on the relative position.

The relative position between the lesion tissue and the thoracic wall side reflects whether the current positioning of the target breast enables all lesion tissues to be scanned. Specifically, in response to determining that the relative position is greater than a distance threshold, it indicates that the lesion tissue is at a certain distance from the thoracic wall side and close to an imaging center, so that all lesion tissues can be scanned, and the position of the lesion tissue does not need to be adjusted; or, in response to determining that the relative position is less than the distance threshold, it indicates that the lesion tissue is too close to the thoracic wall side, a portion of lesion tissues may not be placed on the support paddle and therefore cannot be imaged, and the position of the lesion tissue needs to be adjusted so that all lesion tissues can be scanned. The distance threshold may be set manually or determined based on a scanning blind zone of the breast imaging system 200. Merely by way of example, a larger scanning blind zone corresponds to a larger distance threshold. The scanning blind zone of the breast imaging system 200 refers to a region of the target breast in the pressure distribution map that is beyond an imaging range of the breast scanning device 150.

Merely by way of example, as shown in FIG. 7A, in response to determining that the relative position d between the lesion tissue and the thoracic wall side is less than the distance threshold, it implies that a lesion tissue (such as a mass) may also exist on the right-side of the current lesion tissue. Some lesion tissue would consequently be missed during scanning, indicating that the position of the lesion tissue needs to be adjusted.

In some embodiments of the present disclosure, by assessing whether the current positioning allows for complete lesion scanning based on the initial pressure distribution map, the system prevents situations where positioning errors or incorrect gantry parameters lead to incomplete imaging. This avoids the need for repeat scans and spares the patient unnecessary radiation exposure.

In response to determining that the position of the lesion tissue does not need to be adjusted, the processing device 110 may determine that the current positioning passes the verification and execute operation 530. In response to determining that the position of the lesion tissue needs to be adjusted, the processing device 110 may determine that the current positioning does not pass the verification and execute operation 540.

In operation 530, in response to determining that the current positioning passes the verification, the breast scanning device may be controlled to perform a target scan on the target breast.

In response to determining that the current positioning passes the verification, the processing device 110 may perform the target scan on the target breast based on one or more target scanning parameters (a target compression parameter, a target exposure parameter, and a target scanning angle) in a scanning protocol of the target scan. In some embodiments, if the target compression parameter is different from the initial compression parameter used in operation 510 for obtaining the initial pressure distribution map, the breast scanning device is controlled to re-compress the target breast based on the target compression parameter before performing the target scan. In some embodiments, the scanning protocol of the target scan may be preset. For example, the scanning protocol may be preset by a user.

In some embodiments, the one or more target scanning parameters include the target exposure parameter. The processing device 110 determines the target exposure parameter by performing a process 800 in FIG. 8 and controls the breast scanning device to perform the target scan based on the target exposure parameter. In some embodiments, the processing device 110 designates the initial pressure distribution map as the pressure distribution map after the current positioning passes the verification, obtains a reference identification result based on the pressure distribution map (which will be described in relevant descriptions of FIG. 12), determines the one or more target scanning parameters based on the reference identification result, and controls the breast scanning device to perform the target scan based on the one or more target scanning parameters.

In some embodiments, the processing device 110 further obtains a dynamic pressure distribution map, the dynamic pressure distribution map being acquired by the pressure sensor from the initiation of the compression on the target breast (i.e., when a compression force is applied) until the target breast is in the compressed state (i.e., when the compression force reaches a preset threshold); determines lesion information associated with the target breast based on the dynamic pressure distribution map, the lesion information including lesion position information and benign-malignant status information; and adds an annotation associated with the lesion information to a target scanning image acquired by the target scan for display. More descriptions regarding the dynamic pressure distribution map and the addition of annotation may be found in the related descriptions of operation 360 and a process 1500 in FIG. 15.

In operation 540, in response to determining that the current positioning does not pass the verification, a prompt may be issued to adjust the current positioning.

In some embodiments, the processing device 110 may prompt a user to adjust the current positioning of the target breast via the terminal device 130 through text, voice, vibration, or the like. The positioning adjustment of the target breast may be achieved by adjusting the target breast itself or by adjusting a position of a gantry.

In some embodiments of the present disclosure, verifying the current positioning based on the initial pressure distribution map before performing the target scan on the target breast can reduce the acquisition of invalid images, thereby preventing unnecessary exposure of the patient to radiation, and also avoiding misdiagnosis caused by incorrect positioning.

In some embodiments, the breast scanning device includes a localized compression paddle and a support table. A pressure sensor is provided on a surface of the support table that contacts the target breast. Operation 520 may include following steps: determining a relative position between the lesion tissue of the target breast and the localized compression paddle based on the initial pressure distribution map; and determining whether the position of the lesion tissue needs to be adjusted based on the relative position; in response to determining that the position of the lesion tissue does not need to be adjusted, determining that the current positioning passes the verification; or, in response to determining that the position of the lesion tissue needs to be adjusted, determining that the current positioning does not pass the verification.

The localized compression paddle is configured to compress a local tissue of the target breast. Detailed descriptions regarding the localized compression paddle may be found in FIG. 1, FIG. 18, and related descriptions thereof.

The relative position between the lesion tissue and the localized compression paddle may reflect whether the lesion tissue is within a projection range corresponding to the localized compression paddle in a direction perpendicular to the pressure sensor. For example, as shown in FIG. 18, the processing device 110 may identify a lesion region corresponding to the lesion tissue from the initial pressure distribution map, and determine a projection position 155′ of the lesion tissue on the pressure sensor in the direction perpendicular to the pressure sensor based on a position of the lesion region in the initial pressure distribution map. Detailed descriptions regarding identifying the lesion region corresponding to the lesion tissue from the pressure distribution map may be found in FIG. 8. Then, the processing device 110 may determine a projection range 154′ of the localized compression paddle on the pressure sensor in the direction perpendicular to the pressure sensor based on a relative position between the localized compression paddle 154 and the pressure sensor 153-3. Then, the processing device 110 may determine the relative position between the lesion tissue and the localized compression paddle based on the projection position 155′ of the lesion tissue on the pressure sensor and the projection range 154′ of the localized compression paddle on the pressure sensor. For example, if the projection position 155′ is within the projection range 154′, the processing device 110 determines that the lesion tissue is within the projection range corresponding to the localized compression paddle; otherwise, the processing device 110 determines that the lesion tissue is not within the projection range corresponding to the localized compression paddle.

In some embodiments, the localized compression paddle includes a puncture hole. The relative position between the lesion tissue and the localized compression paddle may include a relative position between the lesion tissue and the puncture hole. The relative position between the lesion tissue and the puncture hole may reflect whether the lesion tissue is within a projection range corresponding to the puncture hole in the direction perpendicular to the pressure sensor. As shown in FIG. 18, the processing device 110 may determine a projection range 154-1′ of the puncture hole on the pressure sensor in the direction perpendicular to the pressure sensor based on a relative position between the puncture hole 154-1 and the pressure sensor 153-3. The processing device 110 then determines the relative position between the lesion tissue and the puncture hole based on the projection position 155′ of the lesion tissue on the pressure sensor and the projection range 154-1′ of the puncture hole on the pressure sensor. For example, if the projection position 155′ is within the projection range 154-1′, the processing device 110 determines that the projection position of the lesion tissue is within the projection range corresponding to the puncture hole; otherwise, the processing device 110 determines that the lesion tissue is not within the projection range corresponding to the puncture hole.

Further, the processing device 110 determines whether the position of the lesion tissue needs to be adjusted based on the relative position between the lesion tissue and the localized compression paddle (or the puncture hole). In the direction perpendicular to the pressure sensor, in response to determining that the lesion tissue is within the projection range corresponding to the localized compression paddle or the puncture hole, the position of the lesion tissue does not need to be adjusted; or, in response to determining that the lesion tissue is not within the projection range corresponding to the localized compression paddle or the puncture hole, the position of the lesion tissue needs to be adjusted.

In some embodiments, the processing device 110 may check both the relative position between the lesion tissue and the thoracic wall side and the relative position between the lesion tissue and the localized compression paddle (or the puncture hole). Or, the processing device 110 may check only one of the above relative positions.

FIG. 8 is a flowchart illustrating a process of breast imaging according to some embodiments of the present disclosure. In some embodiments, the processing device 110 or the breast imaging system 200 may perform a process 800. As shown in FIG. 8, the process 800 includes the following operations.

In operation 810, a pre-scanning image and a pressure distribution map of a target breast may be obtained.

The pre-scanning image refers to an exposure image obtained through a pre-exposure scan performed before the target scan. In some embodiments, the pre-scanning image is obtained by the breast scanning device by performing the pre-exposure scan on the target breast in a compressed state. Specifically, the processing device 110 may control the breast scanning device to compress the target breast based on a target compression parameter corresponding to the target scan, control the breast scanning device to irradiate the target breast in the compressed state with a low dose of X-rays based on a pre-exposure parameter, and acquire the corresponding pre-scanning image. The pre-exposure parameter is an imaging parameter for obtaining the pre-scanning image. A dose of X-rays for the target scan may be determined based on the pre-exposure parameter. Detailed descriptions regarding the pre-exposure parameter may be found in the related descriptions of operation 830.

The pressure distribution map refers to a pressure distribution map acquired simultaneously with the pre-scanning image. The pressure distribution map may reflect a pressure distribution of the target breast in the compressed state while acquiring the pre-scanning image. In some embodiments, the pressure distribution map may be acquired by a pressure sensor during the pre-exposure scan. Specifically, when the breast scanning device irradiates the target breast in the compressed state with the low dose of X-rays, the processing device 110 may simultaneously control the pressure sensor to acquire the pressure distribution map.

In some embodiments, the pre-scanning image and the pressure distribution map are obtained through operations 310-330 in FIG. 3.

In operation 820, one or more target regions corresponding to one or more target tissues in the pre-scanning image may be determined based on the pressure distribution map.

The one or more target tissues may include one or more of an adipose tissue, a glandular tissue, and a lesion tissue. In some embodiments, the one or more target tissues include only the lesion tissue. In some embodiments, the one or more target tissues include the glandular tissue and the lesion tissue. The lesion tissue is a tissue in the target breast where suspected lesions, such as masses, calcifications, structural distortions, or abnormal densities, may exist.

In some embodiments, operation 820 includes operation 822 and operation 824.

In operation 822, one or more target tissues of the target breast in the pressure distribution map may be identified to obtain a reference identification result.

The reference identification result includes one or more reference regions in the pressure distribution map corresponding to the one or more target tissues. For example, the reference identification result includes a glandular identification result, i.e., a reference glandular region corresponding to the glandular tissue. As another example, the reference identification result includes a lesion identification result, i.e., a reference lesion region corresponding to a lesion tissue. As another example, the reference identification result includes both the lesion identification result and the glandular identification result. More descriptions regarding the lesion identification result may be found in FIG. 13A.

In some embodiments, the reference identification result may be determined based on one or more judgment thresholds. The judgment threshold is a pressure value used to distinguish different target tissues (e.g., an adipose tissue, a glandular tissue, and a lesion tissue). The judgment threshold may be a system default value, or may be set manually, or may be determined by the processing device 110 through data analysis. In some embodiments, the one or more judgment thresholds include an adipose threshold, a glandular threshold, and a malignant lesion threshold, wherein the values of the adipose threshold, the glandular threshold, and the malignant lesion threshold increase sequentially. For example, when a pressure value of a pixel point in the pressure distribution map is less than or equal to the adipose threshold, the pixel point belongs to an adipose tissue. When a pressure value of a pixel point in the pressure distribution map is greater than the adipose threshold and less than or equal to the glandular threshold, the pixel point belongs to a glandular tissue. When a pressure value of a pixel point in the pressure distribution map is greater than the glandular threshold and less than or equal to the malignant lesion threshold, the pixel point belongs to a benign lesion tissue. When a pressure value of a pixel point in the pressure distribution map is greater than the malignant lesion threshold, the pixel point belongs to a malignant lesion tissue. The glandular threshold may also be referred to as a lesion threshold or a benign lesion threshold.

In some embodiments, if the one or more target tissues only include the lesion tissue, the processing device 110 may designate pixel points in the pressure distribution map with pressure values greater than the glandular threshold as pixel points in the reference region (i.e., the judgment threshold is the glandular threshold). If the one or more target tissues include the lesion tissue and the glandular tissue, the processing device 110 may designate pixel points in the pressure distribution map with pressure values greater than the adipose threshold as pixel points in the reference region (i.e., the judgment threshold is the adipose threshold).

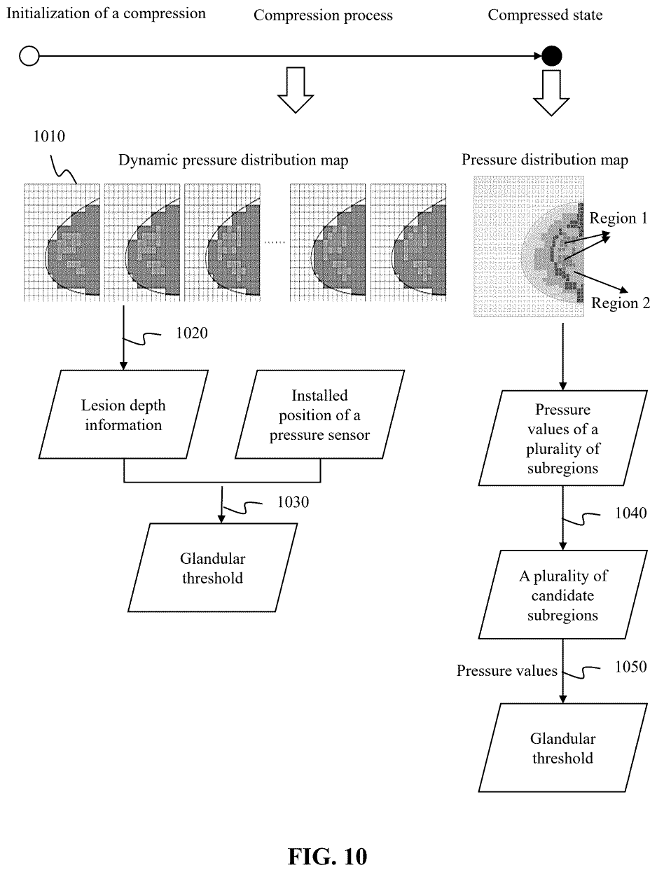

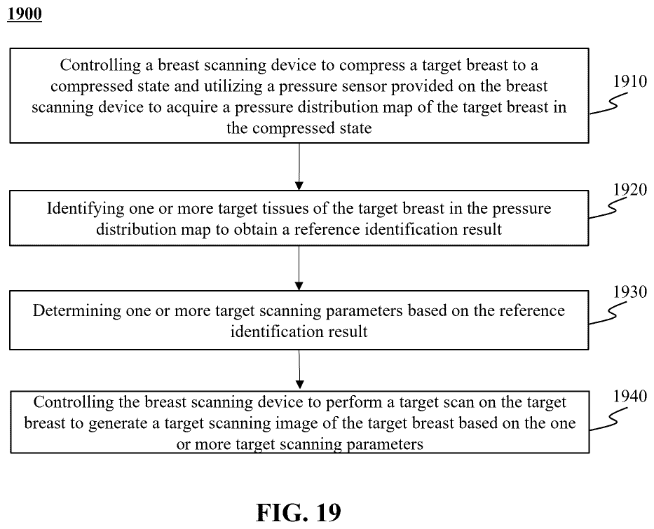

In some embodiments, the glandular threshold may be determined by executing a process in FIG. 10. In some embodiments, the processing device 110 may execute an approach described in operation 1330 of FIG. 13A to determine the glandular threshold. In some embodiments, the processing device 110 may execute an approach described in operation 1920 of FIG. 19 to determine the glandular threshold.

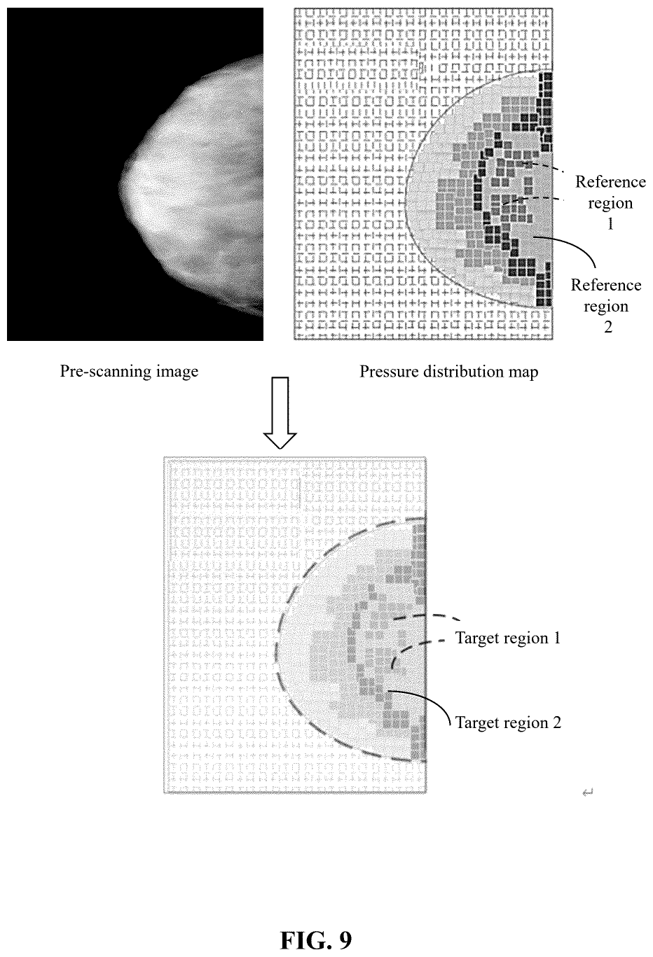

FIG. 9 is a schematic diagram illustrating determining one or more target regions in a pre-scanning image. For example, as shown in FIG. 9, the processing device 110 may determine a plurality of pixel points (or a plurality of subregions) in the pressure distribution map with pressure values greater than or equal to a judgment threshold 1 as pixel points in a reference region 1 (pixel points indicated by dashed lines), thereby obtaining the reference region 1. As another example, as shown in FIG. 9, the processing device 110 may determine a plurality of pixel points (or a plurality of subregions) in the pressure distribution map with pressure values greater than or equal to the judgment threshold 1 as pixel points in the reference region 1 (pixel points indicated by dashed lines), and determine a plurality of pixel points (or a plurality of subregions) in the pressure distribution map with pressure values less than the judgment threshold 1 and greater than or equal to a judgment threshold 2 as pixel points in a reference region 2 (pixel points indicated by solid lines), thereby obtaining the reference region 1 and the reference region 2. For example, the judgment threshold 1 is the malignant lesion threshold and the judgment threshold 2 is the glandular threshold, the reference region 1 is a malignant lesion region, and the reference region 2 is a benign lesion region. In some embodiments, the one or more target tissues may be identified by a user. For example, the pressure distribution map may be displayed to the user, the user may annotate the lesion tissue and/or the glandular tissue in the pressure distribution map.

In operation 824, the one or more target regions in the pre-scanning image may be determined based on the reference identification result. In some embodiments, the one or more target regions may be a target lesion region corresponding to a lesion tissue. In some embodiments, the one or more target regions include a target lesion region corresponding to a lesion tissue and a target glandular region corresponding to a glandular tissue.

As shown in FIG. 9, the processing device 110 may determine corresponding positions of the one or more target regions in the pre-scanning image based on positions of the reference regions in the pressure distribution map, thereby determining the one or more target regions.

In some embodiments of the present disclosure, the reference region(s) in the pressure distribution map can be determined quickly and accurately based on the judgment threshold. Since the pre-scanning image and the pressure distribution map are acquired simultaneously, the target region(s) can be determined quickly and accurately based on the reference region(s), improving accuracy and efficiency in determining the target region(s).

In operation 830, a target exposure parameter may be determined based on the one or more target regions and the pre-exposure parameter corresponding to the pre-scanning image.

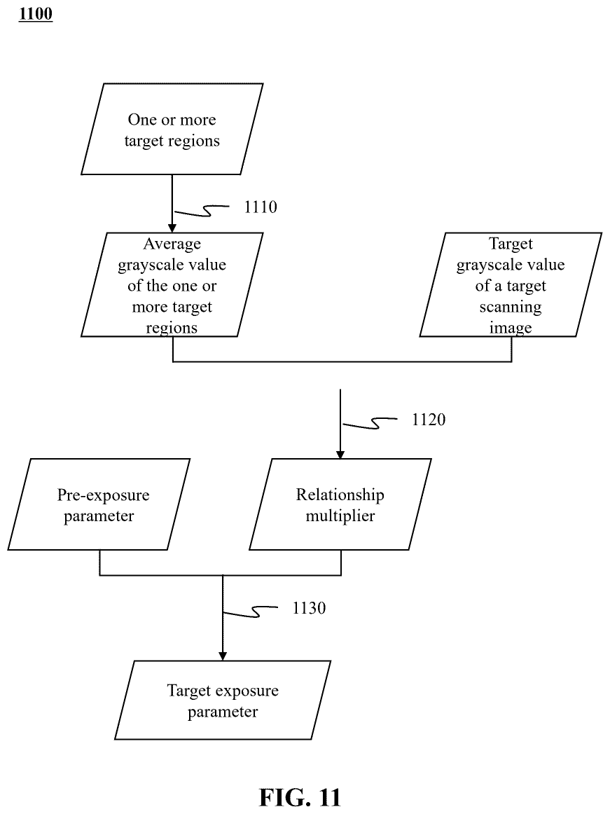

As described above, the pre-exposure parameter is an imaging parameter for obtaining the pre-scanning image, i.e., a scanning parameter of the pre-exposure scan. The target exposure parameter is an imaging parameter for obtaining a target scanning image, i.e., an imaging parameter of the target scan. In some embodiments, the pre-exposure parameter and the target exposure parameter may include a tube current, a tube voltage, a focal spot size, an exposure time, etc. Detailed descriptions regarding determining the target exposure parameter may be found in FIG. 11 and the related descriptions thereof.

In operation 840, the breast scanning device may be controlled to perform a target scan on the target breast based on the target exposure parameter to obtain a target scanning image.

In some embodiments, the processing device 110 may control the breast scanning device to irradiate the target breast in the compressed state with a specific dose of X-rays based on the target exposure parameter and acquire a corresponding target scanning image. The target breast is in the compressed state in the target scan.

In some embodiments of the present disclosure, the one or more target regions in the pre-scanning image may be accurately determined based on the pressure distribution map, and the target exposure parameter is determined based on the one or more target regions in the pre-scanning image, such that the target scanning image can be obtained based on the target exposure parameter with reduced irradiation to the patient while ensuring image quality.

FIG. 10 is a flowchart illustrating a process of determining a glandular threshold according to some embodiments of the present disclosure.

As shown in FIG. 10, the processing device 110 may perform operations 1010-1030 based on a dynamic pressure distribution map to determine the glandular threshold, or perform operations 1040-1050 based on a pressure distribution map to determine the glandular threshold.

In operation 1010, a dynamic pressure distribution map acquired by a pressure sensor during a process of compressing a target breast to a compressed state may be obtained.

As described in operation 810, the processing device 110 may control the breast scanning device to compress the target breast based on the target compression parameter. The dynamic pressure distribution map may reflect a change in pressure distributions over time during a process of compressing the target breast to the compressed state. The dynamic pressure distribution map may include a plurality of frames of pressure distribution maps corresponding to a plurality of times during the compression process.

In some embodiments, the processing device 110 may control the pressure sensor to acquire a plurality of frames of pressure distribution maps at a plurality of times while controlling the compression paddle to compress the target breast based on the target compression parameter. The pressure distribution map may be a last frame of pressure distribution map among the plurality of frames of pressure distribution maps of the dynamic pressure distribution map, and the pressure distribution map reflects a pressure distribution of the target breast in a stable compressed state.

In operation 1020, lesion depth information may be determined based on the dynamic pressure distribution map.

The lesion depth information refers to information reflecting a distance between a lesion tissue and the skin surface of the target breast. The lesion depth information may be represented by a distance between the lesion tissue and the support table, the compression paddle, or the pressure sensor. In some embodiments, the processing device 110 may determine the lesion depth information by executing operation 1710, operation 1731, and operation 1732 in the process 1700.

In operation 1030, the glandular threshold may be determined based on an installed position of the pressure sensor and the lesion depth information.

In some embodiments, the processing device 110 may determine a distance between the lesion tissue and the pressure sensor based on the lesion depth information and the installed position of the pressure sensor, and determine the glandular threshold based on the distance. The smaller the distance between the lesion tissue and the pressure sensor, the easier it is for the pressure sensor to acquire pressure information of the lesion tissue, and the larger the corresponding glandular threshold; or, the larger the distance between the lesion tissue and the pressure sensor, the smaller the corresponding glandular threshold. Merely by way of example, if a mass is located at a relatively upper position of the target breast, when the pressure sensor is provided on a lower surface of the compression paddle, the pressure sensor is relatively close to the mass and receives a relatively large pressure (compared to a case where the pressure sensor is provided on an upper surface of the support table), and the glandular threshold is set to a relatively large value.

In some embodiments of the present disclosure, the lesion depth information is determined based on the dynamic pressure distribution map of the target breast during the compression process, and the glandular threshold is determined based on the installed position of the pressure sensor and the lesion depth information. This approach considers the impact of a position of the lesion tissue within the target breast and the installed position of the pressure sensor on the pressure exerted on the lesion tissue, thereby improving the accuracy of lesion tissue identification.

In operation 1040, a plurality of candidate subregions may be determined based on pressure values of a plurality of subregions in the pressure distribution map.

The pressure distribution map may include a plurality of subregions, and each subregion corresponds to a pressure value. The candidate subregion refers to a subregion used to determine the glandular threshold. In some embodiments, the processing device 110 may determine a subregion with a pressure value greater than a reference glandular threshold as a candidate subregion. The reference glandular threshold may be a manually set threshold with a relatively low value, and the reference glandular threshold may screen out potential lesion tissues.

In operation 1050, a glandular threshold may be determined based on pressure values of the plurality of candidate subregions.

Specifically, the processing device 110 may determine target subregions based on an area and a density of the plurality of candidate subregions. For example, when a plurality of candidate subregions corresponding to the highest pressure peak are relatively large and densely distributed, the plurality of candidate subregions corresponding to the highest pressure peak are determined as the target subregions; or, the plurality of candidate subregions corresponding to the highest pressure peak and a plurality of candidate subregions corresponding to a second-highest pressure peak are determined as the target subregions. Merely by way of example, referring to FIG. 9, assume that the reference region 1 corresponds to the highest pressure peak, and the reference region 2 corresponds to a second-highest pressure peak. If a plurality of candidate subregions in the reference region 1 is relatively large and densely distributed, the plurality of candidate subregions in the reference region 1 are determined as the target subregions. If a plurality of candidate subregions in the reference region 1 is relatively small or dispersed, and a plurality of candidate subregions in the reference region 2 is relatively large and densely distributed, the plurality of candidate subregions in the reference region 1 and the plurality of candidate subregions in the reference region 2 are determined as the target subregions.

Further, the processing device 110 may determine the glandular threshold based on pressure values of the target subregions. For example, an average of the pressure values of the plurality of target subregions is determined as the glandular threshold. As another example, when the target subregion includes regions corresponding to a plurality of pressure peaks, a weighted average of the pressure values of the plurality of target subregions is determined as the glandular threshold. A weight corresponding to each target subregion may be determined based on a ratio of an area of the target subregion to a total area of the plurality of target subregions.

In some embodiments, the processing device 110 may determine a plurality of glandular thresholds based on the pressure values of the plurality of target subregions. For example, the processing device 110 may determine one-third of the total value, the average value, the weighted average value, and two-thirds of the total value of the pressure values of the plurality of target subregions as the plurality of glandular thresholds. As another example, the processing device 110 may determine a corresponding glandular threshold based on an average of pressure values of a plurality of target subregions in each pressure peak region, thereby obtaining a plurality of glandular thresholds corresponding to the plurality of pressure peak regions.

In some embodiments of the present disclosure, the plurality of candidate subregions are determined based on the pressure values of the plurality of subregions, and then the glandular threshold is determined based on the pressure values of the plurality of candidate subregions. This approach allows the glandular threshold to be adaptively adjusted based on the area and density of the plurality of candidate subregions, thereby adapting to different examination requirements and/or various pathological conditions of different breasts.

FIG. 11 is a flowchart illustrating a process of determining a target exposure parameter according to some embodiments of the present disclosure. In some embodiments, the processing device 110 or the breast imaging system 200 may perform a process 1100. As shown in FIG. 11, the process 1100 may include the following operations.

In operation 1110, an average grayscale value of one or more target regions may be determined.

As mentioned above, the target region refers to a region in a pre-scanning image where a glandular tissue and/or a lesion tissue may exist. Specifically, the processing device 110 may acquire grayscale values of a plurality of pixel points in the one or more target regions, and determine the average grayscale value based on the grayscale values of the plurality of pixel points.

In operation 1120, a relationship multiplier may be determined based on the average grayscale value and a target grayscale value of a target scanning image.

The target grayscale value refers to an ideal grayscale value of the target scanning image. The target grayscale value can enable a physician to clearly observe the target region in the target scanning image. The target grayscale value may be a system default value, or may be determined and/or corrected based on a size of the target breast. The relationship multiplier refers to a ratio of the target grayscale value to the average grayscale value.

In operation 1130, a target exposure parameter may be determined based on a pre-exposure parameter corresponding to a pre-scanning image and the relationship multiplier.

In some embodiments, a product of the pre-exposure parameter and the relationship multiplier may be determined as the target exposure parameter.

In some embodiments of the present disclosure, the relationship multiplier is determined based on the average grayscale value of the one or more target regions and the target grayscale value of the target scanning image, and then the target exposure parameter is determined based on the pre-exposure parameter corresponding to the pre-scanning image and the relationship multiplier. This approach enables the target scanning image obtained based on the target exposure parameter to achieve the desired target grayscale value, allowing the physician to clearly visualize the target region.

FIG. 12 is a flowchart illustrating a process of performing a target scan on a target breast according to some embodiments of the present disclosure. In some embodiments, a process 1200 may be performed by the processing device 110. As shown in FIG. 12, the process 1200 includes the following operations.

In operation 1210, a breast scanning device is controlled to compress a target breast to a compressed state and a pressure sensor provided on the breast scanning device is utilized to acquire a pressure distribution map of the target breast in the compressed state.

In some embodiments, the processing device 110 may perform a pre-compression on the target breast based on an initial compression parameter, and obtain a pressure distribution map of the target breast after the pre-compression (the pressure distribution map at this time may also be referred to as an initial pressure distribution map). In some embodiments, the processing device 110 may execute operations 410-430 in FIG. 4 to obtain the pressure distribution map. In some embodiments, if a target compression parameter for the target scan is pre-determined, the breast scanning device may be controlled to compress the target breast based on the target compression parameter to obtain the pressure distribution map.

In operation 1220, a lesion identification result and a breast density of the target breast may be determined based on the pressure distribution map.

In some embodiments, the processing device 110 identifies one or more target tissues of the target breast in the pressure distribution map to obtain a reference identification result. The reference identification result includes an adipose identification result corresponding to an adipose tissue, a glandular identification result corresponding to a glandular tissue, and a lesion identification result corresponding to a lesion tissue. The processing device 110 may further determine the breast density based on the lesion identification result, the glandular identification result, and the adipose identification result. More descriptions regarding determining the breast density may be found in other parts of the present disclosure (e.g., FIG. 13A).

The lesion identification result refers to an identification result of a lesion tissue (e.g., a tumor) of the target breast, and the lesion identification result may include a reference lesion region corresponding to the lesion tissue in the pressure distribution map. In some embodiments, the lesion identification result may further include a lesion type (e.g., calcification, mass) and a lesion position (e.g., a planar position in the target breast, a depth, etc.). The lesion identification result may further include a lesion cycle (e.g., early stage, late stage, etc.), a lesion shape, a lesion size, a lesion distribution, or other related information. More descriptions regarding determining the lesion identification result refer to FIG. 13A and the related description thereof.

The breast density may reflect a density degree or a density level of the target breast, and the breast density may be determined based on components (e.g., adipose tissues, glandular tissues, etc.) of the target breast and a proportion of the components. The breast density includes different types, e.g., a fatty type, a scattered fibroglandular type, a heterogeneously dense type, an extremely dense type, etc. The corresponding breast density increases sequentially from the fatty type, the scattered fibroglandular type, the heterogeneously dense type, and the extremely dense type. Different breast densities may correspond to different scanning parameters (e.g., a scanning angle, etc.).

When a lesion tissue (e.g., a tumor) exists in the target breast, the breast density of the target breast changes. Merely by way of example, when one or more tumors exist in the target breast, different tumors may be distributed at different positions of the target breast, and features of different tumors (e.g., a type, a growth cycle, a shape, a size, etc.) may also be different, thereby causing the breast density of the target breast to change, e.g., changing from the fatty type to the scattered fibroglandular type, the heterogeneously dense type, or the extremely dense type.

In operation 1230, one or more target scanning parameters may be determined based on the lesion identification result and the breast density.

The target scanning parameter refers to a parameter used for the target scan, and the one or more target scanning parameters include at least one of a target compression parameter, a target scanning angle, or a target exposure parameter. Merely by way of example, the target compression parameter may include parameters such as a target compression thickness, a target compression force, etc. The target scanning angle includes parameters such as a scanning angle range or an angle value within the scanning angle range. The target exposure parameter includes parameters such as filtration, tube voltage, milliampere-second, etc. In some embodiments, the one or more target scanning parameters only include the target scanning angle and the target exposure parameter, and the target compression parameter is pre-determined. For example, a preset initial compression parameter may be directly used as the target compression parameter, and in this case, it is unnecessary to determine the target compression parameter.

It should be noted that different target scanning parameters have different effects on a subsequent target scan. For example, for the target compression parameter, different target compression thicknesses and/or different target compression forces affect the quality of a target scanning image, as well as the patient's compression experience (e.g., pain or discomfort). As another example, the target exposure parameter affects a radiation dose to the patient. As another example, for a scanning angle, a small-angle scan can shorten an examination time and reduce the radiation dose, while a large-angle scan can improve image resolution and image quality. Therefore, the target scanning angle can be used to balance the radiation dose and the image quality. As another example, for the target exposure parameter, different target exposure parameters affect the image clarity and the radiation dose to the patient. As another example, when a malignant or large-area lesion exists, a compression force may be appropriately reduced to avoid causing pain to the patient.

In some embodiments, the processing device 110 may determine the target scanning angle, the target compression parameter, and the target exposure parameter based on the breast density and the lesion identification result.

For example, a larger breast density may correspond to a larger target scanning angle, to more accurately identify lesions in the target breast and avoid incorrect identification. As another example, when a lesion exists in the target breast and the lesion is located within a region where glands are concentrated, a larger target scanning angle needs to be set. In some embodiments, the processing device 110 may determine the target scanning angle based on the type of breast density of the target breast and scanning angles corresponding to different types of breast density. For example, a fatty type, a scattered fibroglandular type, a heterogeneously dense type, and an extremely dense type correspond to scanning angles that increase sequentially. As another example, a heterogeneously dense type and an extremely dense type correspond to a first scanning angle, and a fatty type and a scattered fibroglandular type correspond to a second scanning angle, where the first scanning angle is greater than the second scanning angle.

In some embodiments, the processing device 110 may determine the target scanning angle based on the breast density and a scanning parameter mapping relationship between breast densities and scanning angles. The scanning parameter mapping relationship may be in a form of a lookup table or a mapping relationship. The scanning parameter mapping relationship may be determined based on historical medical records or manually set by a user.

In some embodiments, a scanning parameter mapping relationship between the breast density, the lesion identification result, and a reference scanning parameter combination is used to determine the target scan parameter(s). The reference scanning parameter combination includes one or more of a plurality of preset scanning parameters (e.g., a compression thickness, a compression force, a scanning angle, filtration, tube voltage, milliampere-second, etc.). The processing device 110 may retrieve or match a corresponding reference scanning parameter combination from the scanning parameter mapping relationship based on the lesion identification result and the breast density to determine a target scanning parameter combination. In some embodiments, the processing device 110 may recommend the reference scanning parameter combination to a user (e.g., physician) to enable the user to make adjustments based on actual conditions (e.g., patient's age, breast-related information, etc.), thereby improving the user experience.

In some embodiments, the processing device 110 may determine the one or more target scanning parameters using a parameter determination model based on the breast density and the lesion identification result. Specifically, the breast density and the lesion identification result may be input into a parameter determination model, and the parameter determination model may output the one or more target scanning parameters.

In some embodiments, the parameter determination model may include one or more of machine learning models such as a support vector machine (SVM), a convolutional neural network (CNN), a deep neural network (DNN) model, etc.

In some embodiments, the parameter determination model may be obtained through training based on a plurality of fourth training samples with a plurality of fourth training labels. The fourth training sample is a sample breast density and a sample lesion identification result, and the fourth training label is corresponding sample scanning parameter(s). The fourth training samples and the fourth training labels may be obtained based on historical scanning images with good scanning effects. For example, historical scanning parameter(s) of a historical scanning image with a good scanning effect is determined as a sample scanning parameter(s), a corresponding historical breast density and a historical lesion identification result are obtained based on the historical scanning image, and the historical breast density and the historical lesion identification result are determined as a sample breast density and a sample lesion identification result.

Specifically, during training, the sample breast density and the sample lesion identification result may be input into an initial parameter determination model, a value of a loss function is determined based on an output of the initial parameter determination model and the fourth training label, parameters of the initial parameter determination model are iteratively updated based on the value of the loss function until an iteration condition is satisfied. Exemplary iteration conditions include the value of the loss function being less than a threshold, a difference between values of the loss function in two adjacent iterations being less than a threshold, an iteration count exceeding a threshold, etc.

In operation 1240, the breast scanning device may be controlled to perform a target scan on the target breast based on the one or more target scanning parameters.

In some embodiments, the processing device 110 may adjust a compression to the target breast applied by the breast scanning device based on the target compression parameter, and control the breast scanning device to perform the target scan on the target breast based on the target scanning angle and the target exposure parameter. In some embodiments, the processing device 110 may control a compression paddle to adjust the compression thickness and the compression force based on a difference between the target compression parameter and the initial compression parameter to achieve the target compression parameter; or, after obtaining the pressure distribution map, the processing device 110 may control the compression paddle to release the compression on the target breast, and perform a second compression on the target breast after the target compression parameter is determined. Thus, the duration of patient's breast under compression is reduced, thereby increasing the patient's comfort.

In some embodiments, before determining the lesion identification result and the breast density, the processing device 110 may further obtain an initial pressure distribution map and perform a verification on a current positioning of the target breast based on the initial pressure distribution map. In response to determining that the current positioning of the target breast does not pass the verification, a prompt may be issued to adjust the current positioning of the target breast. More descriptions related to positioning verification may be found in FIG. 5 and the related description thereof.

In some embodiments, the processing device 110 may further obtain a target scanning image acquired during the target scan and a dynamic pressure distribution map acquired by the pressure sensor during a process of compressing on the target breast to the compressed state based on the target compression parameter; determine target lesion information associated with the target breast based on the dynamic pressure distribution map, the target lesion information at least including lesion depth information and lesion benign-malignant status information; add an annotation associated with the target lesion information to the target scanning image based on the target lesion information; and display the target scanning image with the annotation using a terminal device. More related content regarding the foregoing embodiments may be found in other parts of the present disclosure (e.g., FIGS. 15, 16A).

In some embodiments of the present disclosure, before the target scan is performed, the lesion identification result and the breast density are determined based on the pressure distribution map, which can avoid pre-exposure and reduce radiation received by the patient. At the same time, this approach can automatically determine one or more target scanning parameters corresponding to the target scan, improving the accuracy and relevance of the parameters, while reducing the workload during the preparation stage and improving the user experience.

FIG. 13A is a flowchart illustrating a process of determining a lesion identification result of a target breast according to some embodiments of the present disclosure. In some embodiments, a process 1300 may be performed by the processing device 110. As shown in FIG. 13A, the process 1300 includes the following operations.

In operation 1310, a pressure distribution map may be segmented into a plurality of subregions.

In some embodiments, the processing device 110 may perform a segmentation process on the pressure distribution map based on a preset segmentation algorithm to obtain the plurality of subregions. Merely by way of example, the segmentation algorithm may include a gridding algorithm, and each grid cell may serve as a subregion. The grid cell may be any shape, such as a square, a rectangle, a hexagon, etc. In some embodiments, the segmentation process may be performed based on a distribution of pressure sensing units in a pressure sensor, and each grid cell corresponds to one pressure sensing unit.

In some embodiments, the processing device 110 may determine a pressure uniformity based on the pressure distribution map; determine a region granularity based on the pressure uniformity; and then segment the pressure distribution map based on the region granularity to determine the plurality of subregions.

The pressure uniformity may be used to reflect the differences in pressure values among different regions in the pressure distribution map. For example, when the tissue composition of the target breast is more homogeneous (e.g., the target breast consists entirely or predominantly of adipose tissues or glandular tissues), differences in pressure values corresponding to different breast regions are smaller, indicating greater uniformity and a larger pressure uniformity. When a distribution of different types of tissues of the target breast is more dispersed, the differences in pressure values among different breast regions are larger, indicating a smaller pressure uniformity.

In some embodiments, the pressure uniformity may be determined based on a breast density of the target breast. For example, for a target breast of a fatty type and a target breast of an extremely dense type, a corresponding pressure uniformity of the pressure distribution map is larger; for a target breast of a scattered fibroglandular type and a target breast of a heterogeneously dense type, a corresponding pressure uniformity of the pressure distribution map is smaller. It can be understood that a smaller pressure uniformity indicates a higher probability that a lesion may exist. In some embodiments, in response to determining that the pressure uniformity is less than a preset uniformity threshold, the processing device 110 may determine that a lesion exists in the target breast. The preset uniformity threshold may be obtained based on the evaluation of experimental data or historical case data. The processing device 110 may localize a lesion and a lesion region based on the pressure uniformity.

The region granularity may be used to reflect a fineness of segmentation, and a larger region granularity indicates a smaller area of each subregion. For example, taking the gridding segmentation algorithm as an example, for a pressure distribution map with a smaller pressure uniformity, the processing device 110 may set a larger number of rows and/or columns to obtain a larger region granularity, thereby obtaining a larger count of subregions.

With reference to FIG. 13B and FIG. 13C, FIG. 13B is a schematic diagram illustrating a pressure distribution map according to some embodiments of the present disclosure. FIG. 13C is a schematic diagram illustrating a segmentation result corresponding to a pressure distribution map according to some embodiments of the present disclosure.

As shown in FIG. 13B, a pressure distribution map 1311 includes a breast region 1301 of a target breast (shown as a semi-elliptical region). The breast region 1301 includes an adipose region 1302 corresponding to an adipose tissue (shown as a white region), a lesion region 1303 corresponding to a lesion tissue (shown as a black area), and a glandular region 1304 corresponding to glandular tissue (shown as a gray area). The tissue composition and lesions (e.g., number and distribution of tumors) of the target breast vary among different patients, and as a result, the pressure distribution map 1311 also differs. The pressure distribution map 1311 includes two lesion regions 1303. The glandular region 1304 may be distributed near the lesion region 1303, or may be distributed in the adipose region 1302.

Different positions (e.g., pixel points of the target breast) or regions in the pressure distribution map 1311 correspond to different pressure values. Referring to FIG. 13C, a segmentation result 1312 corresponding to the pressure distribution map 1311 includes pressure values corresponding to a plurality of subregions. The plurality of subregions are shown as grid cells. It should be noted that specific numerical values of the pressure values in FIG. 13C is merely exemplary. The magnitudes of the numerical values are only used to reflect the differences in pressure values among different subregions. The specific numerical value represents the average of pressure values within each subregion.

Merely by way of example, as shown in the segmentation result 1312, a subregion with a numerical value of 1 represents a subregion corresponding to an adipose tissue; a subregion with a numerical value of 0 represents a subregion outside a breast region; a subregion corresponding to a numerical value between [0,1] represents a boundary of the breast region; a larger numerical value indicates a higher probability that a subregion corresponds to a lesion tissue or glandular tissue.

It can be understood that when performing the segmentation process on the pressure distribution map 1311, a certain subregion may cover one or more of an adipose tissue, a glandular tissue, and a lesion tissue. Therefore, the segmentation result 1312 presents a distribution of different numerical values. When performing the segmentation process on the pressure distribution map 1311, considering the region granularity can improve localization and identification of adipose tissues, glandular tissues, or lesion tissues. As shown in FIG. 13C, when a region granularity is larger, a region covered by a lesion tissue (a region indicated by a circular dashed line) includes a larger count of subregions, and a corresponding boundary is more precise.

In some embodiments of the present disclosure, analyzing the pressure distribution map can better assess the probability and distribution of lesion tissues. Additionally, the pressure distribution map can be segmented into finer regions based on different region granularities, which facilitates the accurate localization of lesion tissues.

In operation 1320, a pressure value of each subregion may be determined based on the pressure distribution map.

In some embodiments, for each subregion, the processing device 110 may determine a pressure value of each subregion based on the average of pressure values corresponding to points within the subregion.

In operation 1330, a lesion identification result of the target breast may be determined based on the pressure value of each subregion.

In some embodiments, the processing device 110 may set a glandular threshold. In response to determining that a pressure value corresponding to a certain subregion is greater than the glandular threshold, the processing device 110 may determine that the subregion belongs to a lesion region. The glandular threshold may be a system default value, or may be set manually, or may be determined by the processing device 110 through data analysis.

In some embodiments, the glandular threshold may be determined based on an average pressure value of the breast region. Merely by way of example, 1.5 times the average pressure value of the breast region in the pressure distribution map may be determined as the glandular threshold. The breast region may be a collection of subregions with pressure values not equal to 0. The average pressure value may be an average value of the pressure values of all subregions located within the breast region.

In some embodiments, the processing device 110 may determine the glandular threshold based on a patient feature such as disease type, breast size, age, etc. The glandular threshold corresponding to patients with different patient features may be different. In some embodiments, the processing device 110 may determine the glandular threshold based on an initial compression parameter and patient information. Lesion regions of patients with different features correspond to different pressure values based on different compression parameters (e.g., a compression thickness, a compression force, etc.). Therefore, the glandular threshold corresponding to the target breast may be determined based on a correspondence between patient features and glandular thresholds and a correspondence between compression parameters and glandular thresholds.

In some embodiments, the processing device 110 may obtain a dynamic pressure distribution map acquired by a pressure sensor during a process of compressing the target breast; determine lesion depth information based on the dynamic pressure distribution map; and determine the glandular threshold based on an installed position of the pressure sensor and the lesion depth information. More descriptions regarding determining the glandular threshold based on the dynamic pressure distribution map may be found in FIG. 10 and descriptions thereof. In some embodiments, the processing device 110 may execute operation 1040 and operation 1050 on the pressure distribution map to determine the glandular threshold.

In some embodiments, the processing device 110 may further determine a glandular identification result and an adipose identification result of the target breast based on the pressure value of each subregion, and determine the breast density of the target breast based on the lesion identification result, the glandular identification result, and the adipose identification result.

The glandular identification result refers to an identification result corresponding to the glandular tissue. The glandular identification result includes a glandular region in the pressure distribution map corresponding to the glandular tissue. The adipose identification result refers to an identification result corresponding to the adipose tissue. The adipose identification result includes an adipose region in the pressure distribution map corresponding to the adipose tissue. In some embodiments, the processing device 110 may determine an adipose threshold in a manner similar to determining the glandular threshold. Merely by way of example, 1 or 1.2 times the average pressure value of the breast region may be determined as the adipose threshold.

For each subregion, in response to determining that a pressure value of the subregion is less than the adipose threshold, the processing device 110 determines that the subregion belongs to an adipose region; in response to determining that the pressure value of the subregion is less than the glandular threshold and greater than the adipose threshold, the processing device 110 determines that the subregion belongs to a glandular region. The glandular threshold is greater than the adipose threshold.

In some embodiments, the processing device 110 may determine an adipose region area, a glandular region area, and a lesion region area based on the adipose identification result, the glandular identification result, and the lesion identification result, respectively. The processing device 110 may then determine the breast density of the target breast based on a ratio of a sum of the glandular region area and the lesion region area to a breast area. The breast area may be a sum of the adipose region area, the glandular region area, and the lesion region area.

In some embodiments of the present disclosure, by segmenting the pressure distribution map to obtain a plurality of subregions and according to a relationship between pressure values of different subregions and different breast tissues and/or lesion tissues, identification results of lesion tissues and breast tissues (e.g., an adipose tissue and a glandular tissue) can be accurately obtained.

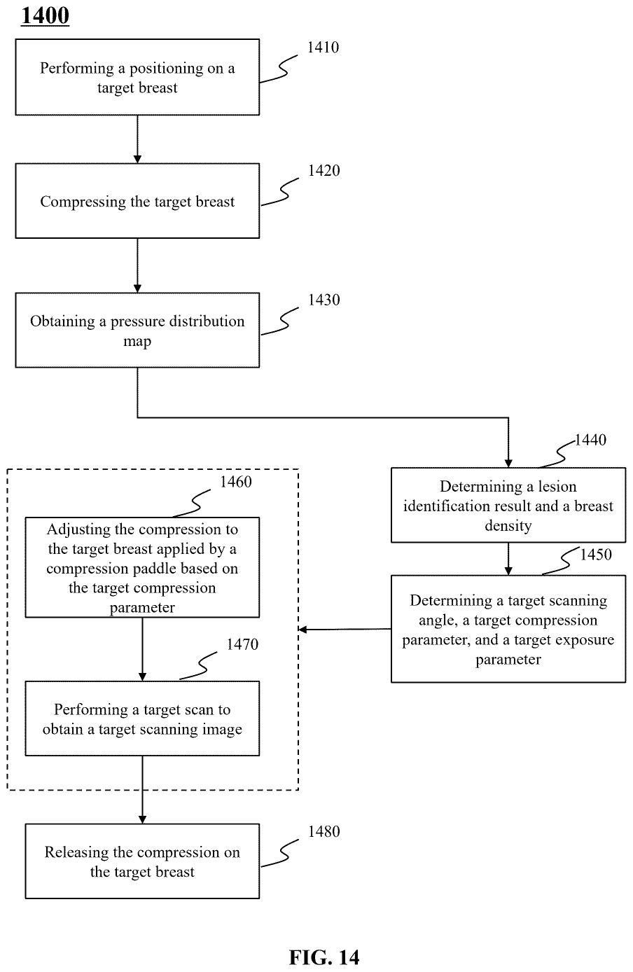

FIG. 14 is a flowchart illustrating another process of performing a target scan on a target breast according to some embodiments of the present disclosure. In some embodiments, a process 1400 may be performed by the processing device 110. As shown in FIG. 1400, the process 1400 includes the following operations.

In operation 1410, a positioning may be performed on a target breast. In some embodiments, the positioning may be performed on the target breast based on a target pose corresponding to a target scan.

In operation 1420, the target breast may be compressed. In some embodiments, the processing device 110 may control a breast scanning device to compress the target breast based on an initial compression parameter.

In operation 1430, a pressure distribution map may be obtained. More descriptions regarding obtaining the pressure distribution map may be found in the description of in operation 1210.

In operation 1440, a lesion identification result and a breast density may be determined based on the pressure distribution map. More descriptions for determining the lesion identification result and the breast density may be found in the description of operation 1220.

In operation 1450, a target scanning angle, a target compression parameter, and a target exposure parameter may be determined. More descriptions regarding determining one or more target scanning parameters may be found in the description of operation 1230.

In operation 1460, the compression to the target breast applied by a compression paddle may be adjusted based on the target compression parameter.

In operation 1470, a target scanning image may be obtained by performing a target scan based on the target scanning angle and the target exposure parameter. More descriptions regarding performing the target scan may be found in the description of operation 1240.

In operation 1480, the compression on the target breast may be released.

After obtaining the target scanning image, the processing device 110 may control the breast scanning device to adjust the compression paddle to release the compression on the target breast.

In some embodiments, the processing device 110 may also analyze and/or process the target scanning image. For example, the processing device 110 may add an annotation to the target scanning image.

FIG. 15 is a flowchart illustrating a process of breast imaging according to some embodiments of the present disclosure. In some embodiments, a process 1500 may be performed by the processing device 110. As shown in FIG. 15, the process 1500 includes the following operations.

In operation 1510, a breast scanning device is controlled to compress a target breast and a pressure sensor provided on the breast scanning device is utilized to acquire a dynamic pressure distribution map of the target breast during a compression process.

Operation 1510 may be executed before performing a target scan. In some embodiments, the processing device 110 controls a compression paddle of the breast scanning device to compress the target breast based on a target compression parameter. The target compression parameter may be determined based on a scanning protocol of the target scan, or may be manually set by a user. In some embodiments, the target compression parameter is determined through operations 1210-1230 in process 1200.

During the compression process, the pressure sensor of the breast scanning device may acquire the dynamic pressure distribution map. The dynamic pressure distribution map includes pressure distribution maps acquired by the pressure sensor at a plurality of times. More descriptions for the pressure sensor, the pressure distribution map, and the dynamic pressure distribution map may be found in other parts of the present disclosure (e.g., FIG. 1).

In operation 1520, the breast scanning device may be controlled to perform a target scan on the target breast in a compressed state to acquire a target scanning image.

After compressing the target breast based on the target compression parameter, the processing device 110 may control the breast scanning device to perform the target scan on the target breast in the compressed state based on a target scanning angle and a target exposure parameter, to acquire the target scanning image. The target scanning angle and the target exposure parameter may be determined based on the scanning protocol of the target scan, or may be manually set by a user. In some embodiments, the target scanning angle and the target exposure parameter are determined through operations 1210-1230 in process 1200. In some embodiments, the target exposure parameter is determined through operations 810-830 in a process 800.

In some embodiments, the processing device 110 may also perform a verification on a current positioning of the target breast based on the dynamic pressure distribution map. In response to determining that the current positioning passes the verification, the processing device 110 controls the breast scanning device to perform the target scan on the target breast; or, in response to determining that the current positioning does not pass the verification, the processing device 110 issues a prompt to adjust the current positioning of the target breast. For example, the processing device 110 may execute operation 520 in process 500 based on any pressure distribution map (e.g., a last frame of pressure distribution map) among the pressure distribution maps of the dynamic pressure distribution map to determine whether the current positioning passes the verification.

In some embodiments, before the target scan, the processing device 110 may determine one or more target scanning parameters based on the dynamic pressure distribution map. The target scan is performed based on the one or more target scanning parameters. For example, the processing device 110 may select any frame of pressure distribution map (e.g., a last frame of pressure distribution map) from the dynamic pressure distribution map, identify one or more target tissues of the target breast in the frame of pressure distribution map to obtain a reference identification result, and determine the one or more target scanning parameters based on the reference identification result. More descriptions for determining the one or more target scanning parameters based on the pressure distribution map may be found in FIG. 9, FIG. 12, and FIG. 19, and are not repeated here.

In operation 1530, lesion information associated with the target breast may be determined based on the dynamic pressure distribution map.

The lesion information includes various types of information associated with a lesion tissue. For example, the lesion information may include a lesion type (e.g., calcification, tumor, or the like), a quantity, a shape, a size (e.g., a dimension, an area, a volume, or the like), lesion position information, and benign-malignant status information, etc. In some embodiments, the lesion information includes at least the lesion position information and the benign-malignant status information.

The lesion position information may include lesion planar position information and lesion depth information. The lesion planar position information represents a position of the lesion tissue in a two-dimensional coordinate system corresponding to the pressure distribution map and/or the target scanning image. The planar position may be represented in a form of coordinates (e.g., two-dimensional coordinates). The lesion depth information may represent a distance (e.g., 5 cm, 10 cm) between the skin surface of the target breast and the lesion tissue (e.g., a center point of the lesion tissue). In some embodiments, the lesion depth information may include a distance between the lesion tissue (e.g., a center point of the lesion tissue) of the target breast and the pressure sensor, the compression paddle, or a support table in a compression direction (i.e., a movement direction of the compression paddle).

The benign-malignant status information may reflect whether the lesion tissue is malignant or benign. For example, the benign-malignant status information may include a classification of the lesion tissue (e.g., a malignant mass and a benign mass).

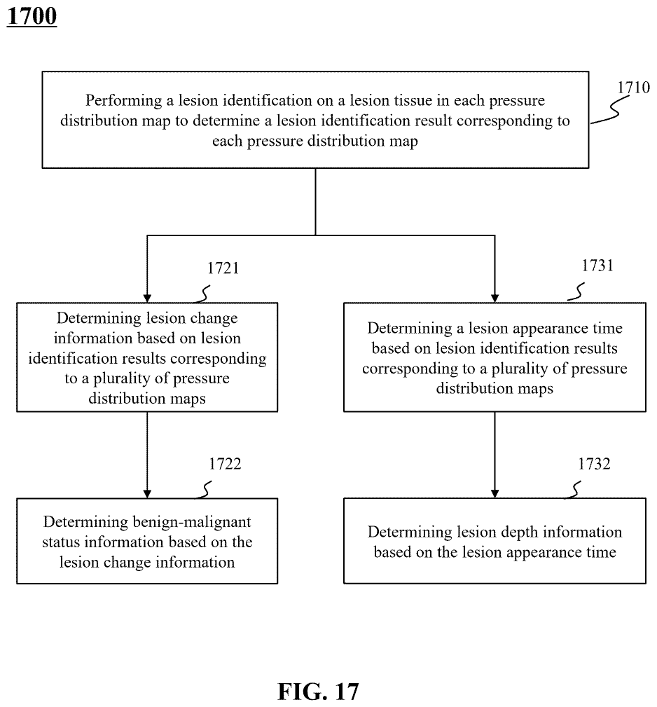

More descriptions for determining the lesion position information and the benign-malignant status information may be found in other parts of the present disclosure (e.g., FIG. 17).

In operation 1540, the target scanning image may be processed based on the lesion information to generate a processed target scanning image.

In some embodiments, the processed target scanning image includes the target scanning image with an annotation or a dual modality fused image.

In some embodiments, the processing device 110 may associate lesion position information of the lesion tissue in the dynamic pressure distribution map with lesion position information of the lesion tissue in the target scanning image. For example, the processing device 110 may determine a target pressure distribution map corresponding to the target scanning image from the dynamic pressure distribution map. The processing device 110 then performs registration processing on location coordinates of the lesion tissue between the target pressure distribution map and the target scanning image, so that location coordinates of the lesion tissue in the target pressure distribution map are aligned with location coordinates of the lesion tissue in the target scanning image. More descriptions regarding the target pressure distribution map may be found in FIG. 16A and FIG. 16B and the descriptions thereof.

In some embodiments, the processing device 110 may add an annotation associated with the lesion information to the target scanning image to generate the processed target scanning image for image display.

The annotation is configured to present relevant information of the lesion tissue to a user. In some embodiments, the annotation may include various forms of annotations such as color annotations (e.g., red, gray, etc.), position annotations (e.g., center point coordinates), text annotations (e.g., textual descriptions), or the like. Merely by way of example, different colors and/or shades of colors (e.g., dark red, light color, etc.) may be used to indicate the benign or malignant status of the lesion tissue.

In some embodiments, the annotation may include a lesion contour annotation. The processing device 110 may determine a lesion contour based on the lesion identification result, and add the annotation associated with the lesion information to the target scanning image based on the lesion contour and the lesion information. The lesion contour may represent a boundary and a range of the lesion tissue. More descriptions regarding the lesion identification result may be found in other parts of the present disclosure (e.g., FIG. 17).

In some embodiments, the processing device 110 may display the target scanning image with the annotation using a terminal device (e.g., the terminal device 130).

In some embodiments of the present disclosure, by combining the advantages of stress imaging and X-ray imaging, the lesion information is determined based on the dynamic pressure distribution map obtained by stress imaging, and the annotation is added to the target scanning image, which can more accurately identify and analyze lesion tissues in the target breast. Meanwhile, more accurate and richer lesion information can be provided to a user (e.g., a physician), which facilitates lesion assessment and improves diagnostic efficiency.

FIG. 16 is a flowchart illustrating a process of displaying a target scanning image according to some embodiments of the present disclosure. In some embodiments, a process 1600 may be performed by the processing device 110. As shown in FIG. 16A, the process 1600 includes the following operations.

In operation 1610, a target pressure distribution map corresponding to a target scanning image may be determined from a dynamic pressure distribution map, a compressed state of a target breast in the target scanning image being consistent with a compressed state of the target breast in the target pressure distribution map.

In the present disclosure, if compression parameters (e.g., compression forces and compression thickness) of the target breast in two images are the same, the compressed state of the target breast in the two images may be considered consistent. Merely by way of example, the target pressure distribution map may be the last frame of pressure distribution map among a plurality of frames of pressure distribution maps of the dynamic pressure distribution map (i.e., a pressure distribution map acquired when a compression is completed). As another example, the target pressure distribution map may be a pressure distribution map acquired simultaneously or nearly simultaneously with the target scanning image. In some embodiments, the target pressure distribution map corresponding to the target scanning image may be directly obtained from the pressure sensor without obtaining the dynamic pressure distribution map.

FIG. 16B is a schematic diagram illustrating a dynamic pressure distribution map according to some embodiments of the present disclosure. As shown in FIG. 16B, a dynamic pressure distribution map 1611 includes a plurality of frames of pressure distribution maps 1611-1 to 1611-n corresponding to a time sequence {T₁, T₂, T₃, . . . , Tₘ, Tₙ}. One of the plurality of frames of pressure distribution maps (e.g., 1611-m) may be referred to as a static pressure distribution map corresponding to a time Tₘ. In some embodiments, a target pressure distribution map may be the last frame of pressure distribution map (e.g., 1611-n).

In operation 1620, a position of a lesion tissue in the target scanning image may be determined based on the target pressure distribution map.

In some embodiments, the processing device 110 may determine a lesion identification result of the lesion tissue based on the target pressure distribution map, and determine the position of the lesion tissue in the target scanning image based on the lesion identification result.

The target scanning image may present adipose tissues, glandular tissues, and lesion tissues of the target breast. The lesion tissue has different shapes and sizes in different growth cycles, which may cause errors in locating and analyzing the lesion tissue in the target scanning image. Merely by way of example, for a small lesion tissue in a target breast with a high breast density (e.g., heterogeneously dense breast, extremely dense breast), glandular tissue may be distributed near the lesion tissue, and the lesion tissue may be visually difficult to distinguish. Therefore, the processing device 110 may locate a lesion region based on differences in pressure values (e.g., pressure force values and pressure intensity values) corresponding to a plurality of different subregions in a breast region obtained from the target pressure distribution map. Merely by way of example, the processing device 110 may determine whether each subregion belongs to a lesion region, thereby accurately locating the lesion region in the target pressure distribution map. In some embodiments, the processing device 110 may determine the lesion identification result based on a method described in operation 1220.

Since the compressed state of the target breast in the target pressure distribution map and the compressed state of the target breast in the target scanning image are consistent, the processing device 110 may determine the position of the lesion tissue in the target scanning image based on the position of the lesion region in the target pressure distribution map. It should be understood that the position of the lesion tissue determined based on the target pressure distribution map is a planar position of the lesion tissue.

In operation 1630, an annotation may be added at the position of the lesion tissue in the target scanning image.

Merely by way of example, a color annotation, a graphic annotation, or a text annotation may be added at the position of the lesion tissue in the target scanning image to display information such as a position of the lesion tissue, a benign or malignant type of the lesion tissue, a boundary of the lesion tissue, or the like. Descriptions regarding the annotation may be found in FIG. 15 and the descriptions thereof, which are not repeated here.

In some embodiments, before determining the position of the lesion tissue in the target scanning image and adding the annotation, the processing device 110 may further perform a size adjustment process on the target pressure distribution map and the target scanning image, so that the size of the target pressure distribution map is the same as the size of the target scanning image. The determination of the position of the lesion tissue in the target scanning image and the addition of the annotation may be performed based on the target pressure distribution map and the target scanning image after the size adjustment process. The size adjustment process may include enlarging and/or reducing the size (e.g., a length and a width) of the target pressure distribution map and/or the size of the target scanning image until the size of the target pressure distribution map is the same as the size of the target scanning image. In some embodiments, the processing device 110 may further perform a registration process on the target pressure distribution map and the target scanning image to align coordinates of the target pressure distribution map and the target scanning image.

In some embodiments, the processing device 110 may execute operation 1640 described below for image display, and/or execute operation 1650 and operation 1660 for image display.

In operation 1640, a terminal device may be controlled to simultaneously display the target pressure distribution map and the target scanning image with the annotation.

Merely by way of example, the target pressure distribution map and the target scanning image with the annotation may be displayed side by side in two columns on the terminal device (e.g., the terminal device 130).

FIG. 16C is a schematic diagram illustrating a target pressure distribution map and a target scanning image with an annotation according to some embodiments of the present disclosure. As shown in FIG. 16C, after a size adjustment process and/or a registration process, the target pressure distribution map 1611-n and a target scanning image 1621 with an annotation are presented side by side on a terminal device. In the target scanning image 1621, the edges and position of a lesion tissue are marked with curves and cross-like symbols.

In operation 1650, the target pressure distribution map and the target scanning image with the annotation may be fused to obtain a dual-modality fused image. Operation 1660, a terminal device may be controlled to display the dual-modality fused image.

FIG. 16D is a schematic diagram illustrating a dual-modality fused image according to some embodiments of the present disclosure.

The dual-modality fused image may be an image generated by overlaying a target pressure distribution map with a target scanning image. The dual-modality fused image includes pressure information of the target pressure distribution map, image information of the target scanning image, and annotation information of the target scanning image. As shown in FIG. 16D, an exemplary dual-modality fused image 1631 is obtained by overlaying a target pressure distribution map corresponding to a target breast with a target scanning image corresponding to the target breast. In the dual-modality fused image 1631, glandular regions or lesion regions are shown in block-like annotations to distinguish them from adipose regions with lower pressure values. In the dual-modality fused image 1631, a lesion region 16311 is represented in a cross-shaped block form. The dual-modality fused image 1631 enables a user (e.g., a physician) to view lesion tissues of the target breast and information of various tissues (e.g., adipose tissues, glandular tissues, etc.) more clearly, thereby facilitating medical diagnosis.

In some embodiments of the present disclosure, displaying the target pressure distribution map and the target scanning image with the annotation side by side or displaying the dual-modality fused image helps users (e.g., physicians) in image interpretation and comparative analysis, providing more accurate guidance for developing treatment plans.

FIG. 17 is a flowchart illustrating a process of determining lesion information associated with a target breast according to some embodiments of the present disclosure. In some embodiments, a process 1700 may be performed by a processing device 110. As shown in FIG. 17, the process 1700 includes the following operations.

In operation 1710, a lesion identification may be performed on a lesion tissue in each pressure distribution map of the dynamic pressure distribution map to determine a lesion identification result corresponding to each pressure distribution map.

The lesion identification result corresponding to the pressure distribution map includes a reference lesion region identified from the pressure distribution map. More descriptions regarding determining the lesion identification result in each pressure distribution map may be found in other parts of the present disclosure (e.g., FIG. 8, FIG. 12, and FIG. 13A).

In some embodiments, the processing device 110 may determine benign-malignant status information based on operation 1721 to operation 1722.

In operation 1721, lesion change information may be determined based on lesion identification results corresponding to the plurality of pressure distribution maps.

The lesion change information may be used to reflect changes in the lesion tissue of the target breast as the compression progresses. In some embodiments, the lesion change information includes first change information of a lesion area and/or second change information of a lesion pressure.

The first change information may be used to reflect a change degree (e.g., magnitude of increase or decrease, etc.) in the lesion area over time across the plurality of pressure distribution maps. Different types of lesions exhibit different hardness, and the deformation degree of the lesions during a process of compressing the target breast differs accordingly. For example, a benign tumor exhibits a smaller hardness compared to a malignant tumor. The benign tumor is more prone to deformation, and the lesion area corresponding to the benign tumor changes more significantly over time. For each pressure distribution map, the processing device 110 may obtain a corresponding lesion area based on the lesion identification result, thereby obtaining the change degree in the lesion area over time across the plurality of pressure distribution maps.

The second change information may be used to reflect a change degree in the lesion pressure over time across the plurality of pressure distribution maps. During a process of compressing the target breast, lesion tissues of different types exhibit different changes in corresponding pressure values. For example, a benign tumor exhibits a smaller hardness compared to a malignant tumor. A pressure value of a lesion region corresponding to the benign tumor changes less over time. For each pressure distribution map, the processing device 110 may obtain an average pressure value of a corresponding reference lesion region based on the lesion identification result, thereby obtaining the change degree in the lesion pressure over time across the plurality of pressure distribution maps.

When there is a plurality of lesion tissues in the target breast, first change information and second change information of each lesion tissue may be determined separately.

In operation 1722, benign-malignant status information may be determined based on the lesion change information.

In some embodiments, the processing device 110 may determine the benign-malignant status information based on the first change information, the second change information, a lesion area change threshold, and a lesion pressure change threshold.

For example, the processing device 110 may determine, based on the first change information, whether an increased magnitude of the lesion area is less than a first change threshold to obtain a first determination result. The processing device 110 may determine, based on the second change information, whether an increased magnitude of the lesion pressure is greater than a second change threshold to obtain a second determination result. The processing device 110 may then determine the benign-malignant status information based on the first determination result and the second determination result.

The first change threshold may be an area change threshold for a malignant lesion, which is used to determine whether the lesion tissue conforms to an area change law of the malignant lesion. The malignant lesion exhibits greater hardness and is less prone to deformation. In response to determining that the increased magnitude of the lesion area is less than the first change threshold, it indicates a higher probability that the lesion tissue is malignant, and the first determination result may be that the lesion tissue is malignant.

The second change threshold may be a pressure change threshold for a malignant lesion, which is used to determine whether the lesion tissue conforms to a pressure change law of the malignant lesion. In response to determining that the increased magnitude of the lesion pressure is greater than the second change threshold, it indicates a higher probability that the lesion tissue is malignant, and the second determination result may be that the lesion tissue is malignant.

It should be noted that area change laws/a pressure change laws of different tissues are different. For example, an area change law/a pressure change law of a benign lesion tissue may be more similar to an area change law/a pressure change law of a glandular tissue, but differ significantly from an area change law/a pressure change law of a malignant lesion tissue. By determining whether the increased magnitude of the lesion area is less than the first change threshold and whether the increased magnitude of lesion pressure is greater than the second change threshold, whether the lesion tissue is malignant can be clearly determined, thereby making the determined benign-malignant status information more accurate.

In some embodiments, the processing device 110 may determine the benign-malignant status information based on a pressure change rate of the lesion tissue. For example, the processing device 110 may set pressure change response relationships corresponding to different types of lesion tissues, and determine the benign-malignant status information according to the pressure change response relationships.

In some embodiments, the pressure change response relationship may be represented by a pressure change curve (e.g., a functional relationship curve). Merely by way of example, a horizontal axis represents time, and a vertical axis represents a pressure value. The processing device 110 may plot a pressure change curve of the lesion tissue based on the dynamic pressure distribution map and the lesion identification results of the pressure distribution maps in the dynamic pressure distribution map. A slope at any time of the pressure change curve represents a pressure change rate. A larger slope indicates a faster change. The processing device 110 may determine the benign-malignant status information based on reference pressure change rates corresponding to different types of lesion tissues. The reference pressure change rates may be determined based on historical medical records. Merely by way of example, the pressure change rates exhibited by different lesion tissues under a reference pressure parameter (e.g., a reference compression thickness, a reference compression force) can be used as the reference pressure change rates.

In some embodiments, the processing device 110 may determine lesion depth information based on the following operation 1731 to operation 1732.

In operation 1731, a lesion appearance time may be determined based on the lesion identification results corresponding to the plurality of pressure distribution maps.

The processing device 110 may arrange the pressure distribution maps identified as including reference lesion regions in chronological order of acquisition, and determine an acquisition time of the earliest acquired pressure distribution map among the plurality of pressure distribution maps as the lesion appearance time. Merely by way of example, a reference lesion region does not appear in a first pressure distribution map and a second pressure distribution map of the dynamic pressure distribution map, and the reference lesion region starts to appear from a third pressure distribution map onward, then an acquisition time of the third pressure distribution map is determined as the lesion appearance time.

In operation 1732, lesion depth information may be determined based on the lesion appearance time.

The lesion tissue may be located at different positions of the target breast (e.g., a center position, an edge position, etc.). During a process of compressing the target breast, the closer the lesion tissue is to the pressure sensor, the faster the pressure sensor senses a pressure value of the lesion tissue, and the faster the reference lesion region appears in the dynamic pressure distribution map. Different lesion depths correspond to different lesion appearance times. An earlier lesion appearance time indicates that the lesion tissue is closer to the pressure sensor. In some embodiments, a distance between the lesion tissue and the pressure sensor, or a distance between the lesion tissue and the compression paddle/the support table, may be determined based on an installed position of the pressure sensor and the lesion appearance time, and this distance may be determined as the lesion depth information.

In some embodiments, the processing device 110 may determine the lesion depth information through a lesion depth reference relationship according to the lesion appearance time. The lesion depth reference relationship may be in a form of a data table, a function, etc., and the lesion depth reference relationship may be obtained by fitting sample data. The sample data may include experimental data (e.g., simulation data and emulation data) or historical case data, etc. Merely by way of example, the sample data may include a sample dynamic pressure distribution map of each sample patient among a plurality of sample patients during a compression process, and a sample lesion appearance time may be obtained from the sample pressure distribution maps, and a sample lesion depth may be determined based on actual lesion information (e.g., a position) detected from the sample patient. Further, a lesion depth reference relationship table may be generated by fitting based on sample compression parameters (e.g., a compression force and a compression thickness), sample lesion appearance times, and sample lesion depth corresponding to the plurality of sample patients. In some embodiments, when generating the lesion depth reference relationship, the sample data may further include related information of the sample patients (e.g., an age, a shape of a breast, a composition ratio of each breast tissue, etc.), thereby making the lesion depth reference relationship more targeted. In an actual application, the processing device 110 may determine the lesion appearance time based on the lesion identification results corresponding to the plurality of pressure distribution maps of the target breast of a target patient, and match a corresponding lesion depth in the lesion depth reference relationship table based on related information of the target patient, a compression parameter, and the lesion appearance time.

In addition, as the compression paddle continues to apply pressure, a shape/an area of a lesion tissue closer to the pressure sensor changes less over time. Conversely, a shape/an area of a lesion tissue farther from the pressure sensor changes more over time. Therefore, in some embodiments, the processing device 110 further combines change information in a shape and an area of the reference lesion regions of the pressure distribution maps over time to determine the lesion depth information.

In some embodiments of the present disclosure, the lesion identification results of the target breast and the change information of the lesion identification results over time (e.g., changes in the lesion area, the lesion appearance time, etc.) can be determined based on the dynamic pressure distribution map, thereby accurately evaluating the benign-malignant status information and the lesion depth information of the lesion tissue, and thus providing users with more abundant and accurate lesion information.

FIG. 19 is a flowchart illustrating a process of breast imaging according to some embodiments of the present disclosure. In some embodiments, the processing device 110 or the breast imaging system 200 may perform a process 1900. As shown in FIG. 19, the process 1900 may include the following operations.

In operation 1910, a breast scanning device may be controlled to compress a target breast to a compressed state, and a pressure distribution map of the target breast in the compressed state may be acquired by a pressure sensor provided on the breast scanning device.

In some embodiments, operation 1910 may be performed in a similar manner to operation 1210, and descriptions thereof are not repeated here. In some embodiments, the processing device 110 may execute operation 310 to operation 330 described in FIG. 3 to obtain the pressure distribution map and a pre-scanning image. In some embodiments, the processing device 110 may execute operation 410 to operation 430 described in FIG. 4 to acquire the pressure distribution map.

In operation 1920, one or more target tissues of the target breast in the pressure distribution map may be identified to obtain a reference identification result.

The one or more target tissues at least include a lesion tissue, and the reference identification result includes a lesion identification result. In some embodiments, the one or more target tissues may further include a glandular tissue and an adipose tissue.

In some embodiments, the processing device 110 may determine the lesion identification result based on a glandular threshold. In some embodiments, the processing device 110 may determine a plurality of subregions in the pressure distribution map and a pressure value of each subregion, determine a plurality of candidate subregions based on pressure values of the plurality of subregions, and then determine the glandular threshold based on a plurality of pressure values of the plurality of candidate subregions. More descriptions about this embodiment may be found in related descriptions of operation 1040 to operation 1050.

In some embodiments, the processing device 110 may acquire a dynamic pressure distribution map acquired by the pressure sensor during a process of compressing the target breast to the compressed state, determine lesion depth information based on the dynamic pressure distribution map, and then determine the glandular threshold based on an installed position of the pressure sensor and the lesion depth information. More descriptions about this embodiment may be found in related descriptions of operation 1010 to operation 1030.

In some embodiments, the processing device 110 may acquire the dynamic pressure distribution map acquired by the pressure sensor during a process of compressing the target breast to the compressed state, determine a first pressure feature and a second pressure feature based on the dynamic pressure distribution map, and process the first pressure feature and the second pressure feature using a threshold determination model to determine the glandular threshold.

The first pressure feature refers to a pressure statistical feature of a static pressure distribution map. Merely by way of example, the first pressure feature may include a pressure mean, a variance, a peak value, a skewness, etc. The processing device 110 may perform statistics and calculation on pressure values of various pixel points in each pressure distribution map in the dynamic pressure distribution map to obtain the first pressure feature.

The second pressure feature refers to a temporal change feature of the dynamic pressure distribution map. Merely by way of example, the second pressure feature may include a pressure change rate, a lesion appearance time, etc. The processing device 110 obtains the second pressure feature by analyzing a change rule of pressure values of the pressure distribution maps in the dynamic pressure distribution map over time. Detailed descriptions about acquiring the dynamic pressure distribution map may be found in related descriptions of operation 1510.

The threshold determination model is a trained machine learning model. Specifically, the first pressure feature and the second pressure feature may be input into the threshold determination model, and the threshold determination model outputs one or more corresponding judgment thresholds. In some embodiments, the threshold determination model may include one or more of a decision tree model, a convolutional neural network (CNN) model, a recurrent neural network (RNN) model, a long short-term memory network (LSTM) model, etc.

In some embodiments, the threshold determination model may be obtained by training based on a plurality of second training samples with a plurality of second training labels. The second training sample includes a sample first pressure feature and a sample second pressure feature. The second training sample may be extracted from historical pressure distribution maps of historical examinees. The second training label includes one or more sample judgment thresholds. In some embodiments, the second training label may be manually annotated by a physician based on a historical pressure distribution map, a historical medical image, and a historical pathology report of a historical patient.

In some embodiments, the second training label may be determined based on a historical scanning image and a historical pressure distribution map. The processing device 110 may obtain a historical lesion region in a historical scanning image. The processing device 110 may perform image segmentation on the historical scanning image to obtain the historical lesion region. Optionally, the processing device 110 may determine the historical lesion region in the historical scanning image based on a corresponding historical pathology report. Further, the processing device 110 may obtain a historical reference region (e.g., a lesion region) corresponding to a historical lesion region in a historical pressure distribution map, and determine the judgment threshold(s) based on the historical reference region. For example, a pressure value at the 20th percentile of the historical reference region may be determined as the glandular threshold, and a pressure value at the 95th percentile of the historical reference region may be determined as the malignant lesion threshold.

Specifically, during training, the second training sample may be input into an initial threshold determination model. A value of a loss function may be determined based on an output of the initial threshold determination model and the second training label. Parameters of the initial threshold determination model may be iteratively updated based on the value of the loss function until an iteration condition is satisfied. Exemplary iteration conditions include the value of the loss function being less than a threshold, a difference in the loss function between two adjacent iterations being less than a threshold, an iteration count exceeding a threshold, or the like.

More descriptions regarding determining the reference identification result may be found in operation 822 and operation 1220.

In some embodiments, the processing device 110 may determine the reference identification result corresponding to the pressure distribution map using a tissue identification model. Specifically, the pressure distribution map may be input into the tissue identification model, and the tissue identification model may output a corresponding reference identification result.

In some embodiments, the tissue identification model may include a machine learning model such as a convolutional neural network (CNN), a bidirectional encoder representations from transformers (BERT), a random forest (RF), or the like.

In some embodiments, the tissue identification model may be obtained by training based on a third training sample and a third training label. The third training sample includes a sample pressure distribution map. The third training sample may be obtained based on a historical pressure distribution map acquired by a pressure sensor. The third training label includes a sample identification result corresponding to a sample pressure distribution map. The third training label may be obtained through manual annotation or may be determined based on a sample scanning image corresponding to the sample pressure distribution map. Specifically, during training, the sample pressure distribution map may be input into an initial tissue identification model. A value of a loss function may be determined based on an output of the initial tissue identification model and the third training label. Parameters of the initial tissue identification model may be iteratively updated based on the value of the loss function until an iteration condition is satisfied. Exemplary iteration conditions include the value of the loss function being less than a threshold, a difference in the loss function between two adjacent iterations being less than a threshold, an iteration count exceeding a threshold, or the like.

In operation 1930, one or more target scanning parameters may be determined based on the reference identification result.

The target scanning parameter refers to a parameter to be used during a target scan. The target scanning parameter(s) include at least one of a target compression parameter, a target scanning angle, or a target exposure parameter. Detailed descriptions of the one or more target scanning parameters may be found in related descriptions of operation 1230.

In some embodiments, the processing device 110 may determine the target exposure parameter based on a pre-scanning image and the reference identification result. Specifically, the processing device 110 may obtain a pre-scanning image of the target breast, determine one or more target regions corresponding to one or more target tissues in the pre-scanning image based on the reference identification result, and determine the target exposure parameter based on the one or more target regions and a pre-exposure parameter corresponding to the pre-scanning image. More descriptions regarding determining the target exposure parameter based on the pre-scanning image may be found in FIG. 8 and the related descriptions thereof.

In some embodiments, the processing device 110 may determine a breast density of the target breast based on the lesion identification result, the glandular identification result, and the adipose identification result. Then, the processing device 110 may determine the one or more target scanning parameters based on the lesion identification result and the breast density. More descriptions regarding determining the breast density and determining the one or more target scanning parameters based on the breast density and the reference identification result may be found in related descriptions of operation 1220 and operation 1230.

In operation 1940, the breast scanning device may be controlled to perform a target scan on the target breast based on the one or more target scanning parameters to acquire a target scanning image of the target breast. The target breast is in the compressed state in the target scan.

Detailed descriptions of operation 1940 may be found in related descriptions of operation 840 and operation 1240.

In some embodiments, the processing device 110 may further obtain a dynamic pressure distribution map, determine lesion information associated with the target breast based on the dynamic pressure distribution map, and add an annotation associated with the lesion information to the target scanning image based on the lesion information. More descriptions of the foregoing embodiments may be found in FIG. 16.

Having thus described the basic concepts, it may be rather apparent to those skilled in the art after reading this detailed disclosure that the foregoing detailed disclosure is intended to be presented by way of example only and is not limiting. Various alterations, improvements, and modifications may appear and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an subject oriented programming language such as Java, Scala, Smalltalk, Eiffel, JADE, Emerald, C++, C #, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lies in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

1. A method for breast imaging, implemented on a computing device comprising at least one processor and at least one storage device, the method comprising:
controlling a breast scanning device to compress a target breast to a compressed state and utilizing a pressure sensor provided on the breast scanning device to acquire a pressure distribution map of the target breast in the compressed state;
obtaining a reference identification result by identifying one or more target tissues of the target breast in the pressure distribution map, the one or more target tissues comprising at least one of a lesion tissue or a glandular tissue;
determining, based on the reference identification result, one or more target scanning parameters; and
controlling, based on the one or more target scanning parameters, the breast scanning device to perform a target scan on the target breast to acquire a target scanning image of the target breast.

2. The method of claim 1, wherein the one or more target scanning parameters comprise a target exposure parameter,
the method further comprises: obtaining a pre-scanning image of the target breast, the pre-scanning image being acquired by performing a pre-scan on the target breast in the compressed state using the breast scanning device;
the target exposure parameter is determined by:
determining, based on the reference identification result, one or more target regions corresponding to the one or more target tissues in the pre-scanning image; and
determining, based on the one or more target regions and a pre-exposure parameter corresponding to the pre-scanning image, the target exposure parameter.

3. The method of claim 2, wherein the determining, based on the one or more target regions and a pre-exposure parameter corresponding to the pre-scanning image, the target exposure parameter comprises:
determining an average grayscale value of the one or more target regions;
determining, based on the average grayscale value and a target grayscale value of the target scanning image, a relationship multiplier;
determining, based on the pre-exposure parameter corresponding to the pre-scanning image and the relationship multiplier, the target exposure parameter.

4. The method of claim 1, wherein the one or more target tissues comprise the glandular tissue, the lesion tissue, and an adipose tissue, and the reference identification result comprises a lesion identification result, a glandular identification result, and an adipose identification result,
the determining, based on the reference identification result, one or more target scanning parameters comprises:
determining, based on the lesion identification result, the glandular identification result, and the adipose identification result, a breast density of the target breast; and
determining, based on the lesion identification result and the breast density, the one or more target scanning parameters, the one or more target scanning parameters comprising at least one of a target compression parameter, a target scanning angle, or a target exposure parameter.

5. The method of claim 4, wherein the controlling, based on the one or more target scanning parameters, the breast scanning device to perform a target scan on the target breast comprises:
adjusting, based on the target compression parameter, a compression to the target breast applied by the breast scanning device;
controlling, based on the target scanning angle and the target exposure parameter, the breast scanning device to perform the target scan on the target breast.

6. The method of claim 1, wherein the identification of the lesion tissue is performed based on a glandular threshold, the glandular threshold being determined by:
determining a plurality of subregions in the pressure distribution map and pressure values of the plurality of subregions;
determining, based on the pressure values of the plurality of subregions, a plurality of candidate subregions from the plurality of subregions; and
determining, based on pressure values of the plurality of candidate subregions, the glandular threshold.

7. The method of claim 1, wherein the identification of the lesion tissue is performed based on a glandular threshold, the glandular threshold being determined by:
obtaining a dynamic pressure distribution map acquired by the pressure sensor during a process of compressing the target breast to the compressed state;
determining, based on the dynamic pressure distribution map, lesion depth information; and
determining, based on an installed position of the pressure sensor and the lesion depth information, the glandular threshold.

8. The method of claim 1, wherein the identification of the lesion tissue is performed based on a glandular threshold, the glandular threshold being determined by:
obtaining a dynamic pressure distribution map acquired by the pressure sensor during a process of compressing the target breast to the compressed state;
determining, based on the pressure distribution map, a first pressure feature;
determining, based on the dynamic pressure distribution map, a second pressure feature;
processing the first pressure feature and the second pressure feature using a threshold determination model to determine the glandular threshold, the threshold determination model being a trained machine learning model.

9. The method of claim 1, wherein before acquiring the pressure distribution map, the method further comprises:
utilizing the pressure sensor to acquire an initial pressure distribution map of the target breast;
performing, based on the initial pressure distribution map, a verification on a current positioning of the target breast; and
in response to determining that the current positioning passes the verification, designating the initial pressure distribution map as the pressure distribution map.

10. The method of claim 1, wherein the method further comprises:
obtaining a dynamic pressure distribution map, the dynamic pressure distribution map being acquired by the pressure sensor during a process of compressing the target breast to the compressed state;
determining, based on the dynamic pressure distribution map, lesion information associated with the target breast, the lesion information comprising lesion position information and benign-malignant status information; and
adding an annotation associated with the lesion information to the target scanning image acquired during the target scan.

11. (canceled)

12. The method of claim 9, wherein the performing a verification on a current positioning of the target breast comprises:
determining, based on the initial pressure distribution map, whether a positioning type of the current positioning is the same as a positioning type of a target positioning corresponding to the target scan;
in response to determining that the positioning type of the current positioning is the same as the positioning type of the target positioning, determining that the current positioning passes the verification; or in response to determining that the positioning type of the current positioning is not the same as the positioning type of the target positioning, determining that the current positioning does not pass the verification.

13-15. (canceled)

16. The method of claim 12, wherein the in response to determining that the positioning type of the current positioning is the same as the positioning type of the target positioning, determining that the current positioning passes the verification further comprises:
in response to determining that the positioning type of the current positioning is the same as the positioning type of the target positioning,
determining, based on the initial pressure distribution map, a relative position between a lesion tissue of the target breast and a thoracic wall side;
determining, based on the relative position, whether the position of the lesion tissue needs to be adjusted;
in response to determining that the position of the lesion tissue does not need to be adjusted, determining that the current positioning passes the verification.

17. The method of claim 9, wherein the breast scanning device comprises a localized compression paddle and a support table, the pressure sensor is disposed on a surface of the support table that is in contact with the target breast, the performing a verification on a current positioning of the target breast comprises:
determining, based on the initial pressure distribution map, a relative position between a lesion tissue of the target breast and the localized compression paddle;
determining, based on the relative position, whether the position of the lesion tissue needs to be adjusted; and
in response to determining that the position of the lesion tissue does not need to be adjusted, determining that the current positioning passes the verification; or in response to determining that the position of the lesion tissue needs to be adjusted, determining that the current positioning does not pass the verification.

18-20. (canceled)

21. The method of claim 10, wherein the adding an annotation associated with the lesion information to a target scanning image acquired during the target scan comprises:
determining a target pressure distribution map corresponding to the target scanning image from the dynamic pressure distribution map, a compressed state of the target breast in the target scanning image being consistent with a compressed state of the target breast in the target pressure distribution map;
determining, based on the target pressure distribution map, a position of a lesion tissue in the target scanning image; and
adding the annotation at the position of the lesion position tissue in the target scanning image.

22. (canceled)

23. The method of claim 21, wherein the method further comprises:
fusing the target pressure distribution map and the target scanning image with the annotation to obtain a dual-modality fused image; and
controlling a terminal device to display the dual-modality fused image.

24. The method of claim 10, wherein the dynamic pressure distribution map comprises a plurality of pressure distribution maps, the benign-malignant status information is determined by:
for each pressure distribution map, identifying a lesion tissue in the pressure distribution map to determine a reference lesion region in the pressure distribution map;
determining, based on the reference lesion regions in the plurality of pressure distribution maps, lesion change information; and
determining, based on the lesion change information, the benign-malignant status information.

25. The method of claim 24, wherein the lesion change information comprises at least one of first change information of a lesion area over time or second change information of a lesion pressure over time.

26. The method of claim 10, wherein the dynamic pressure distribution map comprises a plurality of pressure distribution maps, the lesion position information further comprises lesion depth information, and the lesion depth information is determined by:
for each pressure distribution map, identifying a lesion tissue in the pressure distribution map to determine a reference lesion region in each pressure distribution map;
determining, based on the reference lesion regions in the plurality of pressure distribution maps, a lesion appearance time;
determining, based on the lesion appearance time, the lesion depth information.

27-28. (canceled)

29. A system, comprising:
at least one storage device storing a set of instructions for breast imaging; and
at least one processor configured to communicate with the at least one storage device, wherein when executing the set of instructions, the at least one processor is configured to direct the system to perform operations including:
controlling a breast scanning device to compress a target breast to a compressed state and utilizing a pressure sensor provided on the breast scanning device to acquire a pressure distribution map of the target breast in the compressed state;
obtaining a reference identification result by identifying one or more target tissues of the target breast in the pressure distribution map, the one or more target tissues comprising at least one of a lesion tissue or a glandular tissue;
determining, based on the reference identification result, one or more target scanning parameters; and
controlling, based on the one or more target scanning parameters, the breast scanning device to perform a target scan on the target breast to acquire a target scanning image of the target breast.

30. A non-transitory computer readable medium, comprising a set of instructions for breast imaging, wherein when executed by at least one processor, the set of instructions direct the at least one processor to effectuate a method, the method comprising:
controlling a breast scanning device to compress a target breast to a compressed state and utilizing a pressure sensor provided on the breast scanning device to acquire a pressure distribution map of the target breast in the compressed state;
obtaining a reference identification result by identifying one or more target tissues of the target breast in the pressure distribution map, the one or more target tissues comprising at least one of a lesion tissue or a glandular tissue;
determining, based on the reference identification result, one or more target scanning parameters; and
controlling, based on the one or more target scanning parameters, the breast scanning device to perform a target scan on the target breast to acquire a target scanning image of the target breast.

31-34. (canceled)
